(19) United States
(12) Patent Application Publication
(10) Pub. No.: US 2016/0047817 A1
(43) Pub. Date: Feb. 18, 2016

(54) LTBP2 AS A BIOMARKER FOR EVALUATING THE RISK OF DEATH IN A DISEASED SUBJECT

(71) Applicant: MyCartis NV, Gent (BE)

(72) Inventor: Koen Kas, Schilde (BE)

(21) Appl. No.: 14/884,658

(22) Filed: Oct. 15, 2015

Related U.S. Application Data
(62) Division of application No. 13/749,559, filed on Jan. 24, 2013.
Application No. 13/749,559 is a continuation-in-part of application No. 13/072,241, filed on Mar. 25, 2011, now abandoned.
(60) Provisional application No. 61/318,064, filed on Mar. 26, 2010.

(30) Foreign Application Priority Data
Mar. 26, 2010 (EP) 10158061.1

(51) Int. Cl. G01N 33/68

(52) U.S. Cl. CPC G01N 33/68

(57) ABSTRACT

The application discloses methods for treating a subject presenting with one or more signs of an inflammatory condition, or methods for evaluating the risk of death within a year for a subject presenting with one or more signs of an inflammatory condition, based on measuring the quantity of LTBP2 in a sample from the subject; and kits and devices for measuring LTBP2 and/or performing said methods.

### RELATED APPLICATIONS

This application is a divisional of U.S. application Ser. No. 13/749,559, filed Jan. 24, 2013, which is a continuation-in-part of U.S. application Ser. No. 13/072,241, filed Mar. 25, 2011 which claims priority to European provisional application 10158061.1 and U.S. provisional patent application No. 61/318,064, both filed on Mar. 26, 2010. All of the above applications are incorporated herein by reference.

### FIELD OF THE INVENTION

The invention relates to protein- and/or peptide-based biomarkers useful for evaluating the risk of death within a given time interval for a diseased subject; and to related methods, kits and devices.

### BACKGROUND OF THE INVENTION

In many diseases and conditions, a favourable outcome of prophylactic and/or therapeutic treatments is strongly correlated with early and/or accurate prediction, diagnosis, prognosis and/or monitoring of a disease or condition. Therefore, there exists a continuous need for additional and preferably improved manners for early and/or accurate prediction, diagnosis, prognosis and/or monitoring of diseases and conditions to guide the treatment choices.

Patients often present themselves in emergency departments (ED) with symptoms of an inflammatory condition such as undiagnosed shortness of breath, fever, cough, increased respiratory rate, etc. Unfortunately, these symptoms are neither sensitive nor specific and are related to a whole array of possible underlying pathologies ranging from anxiety and hyperventilation to life-threatening causes such as for instance acute heart failure, renal dysfunction, pulmonary diseases, or sepsis. Because early clinical decision making is often critical for patient outcome, tools are necessary for determining which patients are at increased risk of death in order to facilitate early intervention.

Reliable and preferably early detection of an increased risk of death in a subject presenting with one or more signs of an inflammatory condition is critical to effective treatment of such subjects. Consequently, provision of further, alternative and preferably improved markers and tools for the prediction of mortality in such subjects continues to be of prime importance.

The present invention addresses the above needs in the art by identifying biomarkers for evaluating the risk of death within a given time interval in a diseased subject and providing uses therefore.

### SUMMARY OF THE INVENTION

As shown in the examples, the inventors have found that latent transforming growth factor beta binding protein 2 (LTBP2) levels upon admission in subjects manifesting with acute dyspnea were significantly higher in those subjects who will have died within one year post-admission compared to those subjects who will have remained alive at one year. This distinction was also observed when the patient population was divided based on the presence or absence of acute heart failure (AHF), or based on renal (dys)function as measured by GFR. Consequently, the inventors have realised LTBP2 as a new biomarker advantageous for predicting or prognosticating mortality in patients with dyspnea, particularly acute dyspnea, in patients with AHF and/or in patients with renal dysfunction, particularly chronic renal dysfunction.

Hence, provided is a method for predicting or prognosticating mortality in a subject having dyspnea and/or acute heart failure and/or renal dysfunction, comprising measuring the quantity of LTBP2 in a sample from said subject. Preferably, the dyspnea may be acute dyspnea. Preferably, the renal dysfunction may be chronic renal dysfunction, particularly chronic kidney disease. Without limitation, the dyspnea may be associated with or caused by AHF and/or by renal dysfunction; or the dyspnea may be associated with our caused by conditions other than AHF and renal dysfunction; or the subject may have AHF and/or renal dysfunction without dyspnea symptoms.

In an embodiment, the method for predicting or prognosticating mortality in a subject having dyspnea and/or acute heart failure and/or renal dysfunction comprises the steps of: (i) measuring the quantity of LTBP2 in a sample from the subject; (ii) comparing the quantity of LTBP2 measured in (i) with a reference value of the quantity of LTBP2, said reference value representing a known prediction or prognosis of mortality; (iii) finding a deviation or no deviation of the quantity of LTBP2 measured in (i) from the reference value; and (iv) attributing said finding of deviation or no deviation to a particular prediction or prognosis of mortality in the subject.

In certain embodiments, the methods as taught herein may comprise the step of obtaining a biological sample from the subject.

The present methods for predicting or prognosticating mortality may be preferably performed for a subject once the subject presents with or is diagnosed with dyspnea and/or acute heart failure and/or renal dysfunction, more preferably upon the initial (first) presentation or diagnosis of said diseases and conditions.

As shown in the experimental section, increased mortality rate in populations of dyspneic and/or AHF and/or renal failure subjects is associated with elevated levels of LTBP2. Consequently, prediction or prognostication of increased mortality (increased risk or chance of death within a predetermined time interval) can in particular be associated with an elevated level of LTBP2.

Having conducted extensive experiments and testing, the inventors have found that in patients presenting with signs of systemic inflammatory response syndrome (SIRS) or with suspicion of sepsis, LTBP2 levels were significantly higher in non-survivors versus survivors at 28 days following blood culture.

Having further conducted extensive experiments and tests, the inventors have also found that levels of LTBP2 are closely indicative of death in subjects presenting themselves with dyspnea. Especially death due to lung complications was highly correlated to LTBP2 levels in the blood of the subject. In particular, in clinical samples from 299 patients LTBP2 showed a significant association with several tested clinical parameters related to pulmonary injury, in particular pulmonary inflammation.

It shall be appreciated that finding of increased mortality or risk of death in a subject can guide therapeutic decisions to treat the subject's diseases or conditions.

Hence, in a first aspect, the invention relates to a method for treating a subject presenting with one or more signs of an inflammatory condition, preferably a subject having an inflammatory condition, said method comprising the steps of:
(i) obtaining a biological sample from the subject;
(ii) measuring the quantity of latent transforming growth factor beta binding protein 2 (LTBP2) in the sample;
(iii) comparing the quantity of LTBP2 measured in (ii) with a reference value of the quantity of LTBP2, said reference value representing a known risk of death such as a known risk of death within a year for a subject having an inflammatory condition;
(iv) predicting an increased risk of death within a year, for example within about 6 months, within about 5 months, within about 4 months, within about 3 months, within about 2 months, or within about one month, for the subject if the quantity of LTBP2 measured in (ii) substantially corresponds to a reference value representing a subject having an inflammatory condition which will decease within a year, for example within about 6 months, within about 5 months, within about 4 months, within about 3 months, within about 2 months, or within about one month, or if the quantity of LTBP2 measured in (ii) is elevated compared with a reference value representing a subject having an inflammatory condition which will survive within a year, for example within about 6 months, within about 5 months, within about 4 months, within about 3 months, within about 2 months, or within about one month;
(vi) inferring from said increased risk of death within a year, for example within about 6 months, within about 5 months, within about 4 months, within about 3 months, within about 2 months, or within about one month, for the subject, a need for a therapeutic treatment or intervention in the subject; and
(vii) performing a therapeutic treatment or intervention in the subject, for instance administering to the subject a therapeutically effective amount of an active pharmaceutical ingredient capable of decreasing the risk of death. In certain embodiments, the therapeutic treatment or intervention may also be performed by changing the therapeutic treatment, by the addition of goal-directed therapy, by closer monitoring of the subject, or by instalment of a more aggressive therapy.

Examples of active pharmaceutical ingredients capable of decreasing the risk of death in a subject presenting with one or more signs of an inflammatory condition may include, without limitation, anti-microbial agents, preferably anti-bacterial agents, such as antibiotics; analgesics; antipyretics; and anti-inflammatory drugs, such as non-steroidal anti-inflammatory drugs (NSAID). Any one or a combination of two or more may be used.

In certain embodiments, the subject presenting with one or more signs of an inflammatory condition may have sepsis or systemic inflammatory response syndrome (SIRS). In certain embodiments, the subject presenting with one or more signs of an inflammatory condition may have pulmonary inflammation. In certain embodiments, the subject presenting with one or more signs of an inflammatory condition may have undiagnosed acute dyspnea, acute heart failure, or renal dysfunction.

In certain embodiments of the methods as taught herein, the subject may be a critically ill patient.

In certain further embodiments of the methods as taught herein, the subject may be known or suspected to have an inflammatory condition, or the subject may have an inflammatory condition, such as sepsis, SIRS, or pulmonary inflammation.

In certain embodiments of the methods as taught herein, the subject may be presenting with one or more signs of a systemic inflammatory condition, the subject may be known or suspected to have a systemic inflammatory condition, or the subject may have a systemic inflammatory condition such as sepsis, SIRS, or pulmonary inflammation.

In certain embodiments, the methods as defined herein may be for evaluating the risk of death within about 6 months, within about 5 months, within about 4 months, within about 3 months, within about 2 months, or within about one month. In certain preferred embodiments, the methods as defined herein may be for evaluating the risk of death within about one month such as within 4 weeks or 28 days or within 30 days.

In certain embodiments, the methods may be for treating a subject presenting with one or more signs of sepsis, SIRS, pulmonary inflammation, undiagnosed acute dyspnea, acute heart failure, or renal dysfunction, preferably a subject presenting with one or more signs of sepsis, SIRS, or pulmonary inflammation.

A further aspect relates to a method for evaluating the risk of death within a year, for example within about 6 months, within about 5 months, within about 4 months, within about 3 months, within about 2 months, or within about one month, for a subject presenting with one or more signs of an inflammatory condition, preferably a subject having an inflammatory condition, said method comprising the steps of:
(i) obtaining a biological sample from the subject;
(ii) measuring the quantity of LTBP2 in said sample using an immunoassay or using a binding agent capable of specifically binding to LTBP2;
(iii) comparing the quantity of LTBP2 measured in (ii) with a reference value of the quantity of LTBP2, said reference value representing a known risk of death, such as a known risk of death within a year, for example within about 6 months, within about 5 months, within about 4 months, within about 3 months, within about 2 months, or within about one month, for a subject having an inflammatory condition;
(iv) predicting an increased risk of death within a year in the subject if the quantity of LTBP2 measured in (ii) substantially corresponds to a reference value representing a subject having an inflammatory condition which will decease within a year, for example within about 6 months, within about 5 months, within about 4 months, within about 3 months, within about 2 months, or within about one month, or if the quantity of LTBP2 measured in (ii) is elevated compared with a reference value representing a subject having an inflammatory condition which will survive within a year, for example within about 6 months, within about 5 months, within about 4 months, within about 3 months, within about 2 months, or within about one month.

In certain embodiments, the immunoassay may employ an aptamer and/or antibody specifically binding to LTBP2. In certain further embodiments, the binding agent capable of specifically binding to LTBP2 may be an aptamer or antibody specifically binding to LTBP2.

In certain embodiments of the methods as taught herein, the subject presenting with one or more signs of an inflammatory condition may have sepsis or systemic inflammatory response syndrome (SIRS). In certain embodiments of the methods as taught herein, the subject presenting with one or more signs of an inflammatory condition may have pulmonary inflammation. In certain embodiments of the methods as taught herein, the subject presenting with one or more signs of an inflammatory condition may have acute dyspnea, acute heart failure, or renal dysfunction.

In certain embodiments of the methods as taught herein, the subject may be a critically ill patient.

In certain further embodiments of the methods as taught herein, the subject may be known or suspected to have an inflammatory condition, or the subject may have an inflammatory condition, such as sepsis, SIRS, or pulmonary inflammation.

In certain preferred embodiments, the methods as taught herein may be for evaluating the risk of death within about 6 months, within about 5 months, within about 4 months, within about 3 months, within about 2 months, or within about one month. In certain preferred embodiments, the methods as defined herein may be for evaluating the risk of death within about one month such as within 4 weeks or 28 days or within 30 days.

In certain embodiments, the methods may be for evaluating the risk of death within a year, for example within about 6 months, within about 5 months, within about 4 months, within about 3 months, within about 2 months, or within about one month, for a subject presenting with one or more signs of sepsis, SIRS, pulmonary inflammation, undiagnosed acute dyspnea, acute heart failure, or renal dysfunction, preferably for a subject presenting with one or more signs of sepsis, SIRS, or pulmonary inflammation.

In certain embodiments, the methods as taught herein may be used for assessing the risk of dying from a pulmonary cause or complication in the subject. In certain further embodiments, the methods as taught herein may be used for the prognosis that the inflammatory condition will result in death of the subject or not.

In a further aspect the invention relates to a system for evaluating the risk of death within a year, for example within about 6 months, within about 5 months, within about 4 months, within about 3 months, within about 2 months, or within about one month, for a subject presenting with one or more signs of an inflammatory condition, preferably a subject having an inflammatory condition, said system comprising:
a computer data repository that comprises a reference value of the quantity of LTBP2, said reference value representing a known risk of death, preferably a known risk of death within a year, for example within about 6 months, within about 5 months, within about 4 months, within about 3 months, within about 2 months, or within about one month for a subject having an inflammatory condition; and
a computer system programmed to access the data repository and to use information from the data repository in combination with information on the quantity of LTBP2 in a sample from a subject presenting with one or more signs of an inflammatory condition, to make an evaluation of the risk of death within a year, for example within about 6 months, within about 5 months, within about 4 months, within about 3 months, within about 2 months, or within about one month for the subject.

Related embodiments of the invention concern a method for evaluating the risk of death within a year, for example within about 6 months, within about 5 months, within about 4 months, within about 3 months, within about 2 months, or within about one month for a subject presenting with one or more signs of an inflammatory condition, preferably a subject having an inflammatory condition, said method comprising the steps of:
(i) receiving data representative of values of the quantity of LTBP2 in a sample from the subject;
(ii) accessing a data repository on a computer, said data repository comprising a reference value of the quantity of LTBP2, said reference value representing a known risk of death, preferably a known risk of death within a year, for example within about 6 months, within about 5 months, within about 4 months, within about 3 months, within about 2 months, or within about one month for a subject having an inflammatory condition; and
(iii) comparing the data as received in (i) with the reference value in the data repository on the computer, thereby making an evaluation of the risk of death within a year, for example within about 6 months, within about 5 months, within about 4 months, within about 3 months, within about 2 months, or within about one month for the subject.

In certain embodiments, the determination of what action is to be taken, e.g., by a clinician, in view of said evaluation of the risk of death is performed by a (the) computer. In certain embodiments, a (the) computer reports (i.e., generates an electronic report of) the action to be taken, preferably substantially in real time. The action(s) to be taken by a clinician in view of said evaluation of the risk of death may be one or more of administering to the subject a therapeutically effective amount of an active pharmaceutical ingredient capable of decreasing the risk of death, changing the therapeutic treatment, addition of goal-directed therapy, closer monitoring of the subject, or instalment of a more aggressive therapy.

In certain embodiment, the method for monitoring a change in the risk of death in a subject presenting with one or more signs of an inflammatory condition may comprise the steps of: (i) obtaining biological samples from the subject from two or more successive time points; (ii) measuring the quantity of LTBP2 in the samples from said two or more successive time points, whereby the risk of death in the subject is determined at said two or more successive time points; (iii) comparing the quantity of LTBP2 between the samples as measured in (ii); (iv) monitoring a changed risk of death in the subject if the LTBP2 quantity deviates between the samples as compared in (iii).

For example but without limitation, an elevated quantity (i.e., a deviation) of LTBP2 in a sample from a subject compared to a reference value representing the prediction prognosis of a given mortality (i.e., a given, such as a normal, risk or chance of death within a predetermined time interval) indicates that the subject has a comparably greater risk of deceasing within said time interval.

Without limitation, mortality may be suitably expressed as the chance of a subject to decease within an interval of for example several days, several months or several years from the time of performing a prediction or prognostication method, e.g., within about 14 days or more such as within about 21 days or about 28 days or within about 1 month or more such as within about 2 months, about 3 months, about 4 months, about 5 months or within about 6 months or within about 1 year or within about 2, about 3, about 4, about 5, about 6, about 7, about 8, about 9 or about 10 years from the time of performing the prediction or prognostication method.

In an exemplary but non-limiting experiment LTBP2 levels provided satisfactory discrimination between normal and increased mortality in dyspnea, in AHF, and in renal dysfunction subjects when the time interval for considering the alive vs. dead status was set at 1 year from the time of performing the prediction or prognostication method. Hence, in embodiments mortality may be suitably expressed as the chance of a subject to decease within an interval of between 6 months and 2 years and preferably within 1 year from performing the prediction or prognostication method.

In an exemplary but non-limiting experiment, LTBP2 levels provided satisfactory discrimination between normal and increased risk of death in subjects presenting with one or more signs of an inflammatory condition, in particular sepsis or SIRS, when the time interval for considering the alive vs. dead status was set between 2 weeks and 6 weeks, preferably at 4 weeks or at 28 days, i.e. about one month from the time of performing the method as taught herein.

In a further exemplary but non-limiting experiment, LTBP2 levels provided satisfactory discrimination between normal and increased risk of death in subjects presenting with one or more signs of an inflammatory condition, in particular pulmonary inflammation, when the time interval for considering the alive vs. dead status was set between 2 weeks and 6 weeks, preferably at 30 days, i.e. about one month from the time of performing the method as taught herein.

Hence, in certain embodiments, death may be suitably expressed as the chance of a subject to decease within an interval of between 14 days and 6 months and preferably within about one month such as within 28 days or 30 days from performing the method as taught herein.

Any one prediction, diagnosis, prognosis and/or monitoring method as taught herein may preferably allow for sensitivity and/or specificity (preferably, sensitivity and specificity) of at least 50%, at least 60%, at least 70% or at least 80%, e.g., ≧85% or ≧90% or ≧95%, e.g., between about 80% and 100% or between about 85% and 95%.

Reference throughout this specification to “diseases and/or conditions” encompasses any such diseases and conditions as disclosed herein insofar consistent with the context of such a recitation, in particular but without limitation including increased mortality of subjects having dyspnea and/or acute heart failure and/or renal dysfunction, increased risk of death of subjects presenting with one or more signs of an inflammatory condition such as sepsis, SIRS, or pulmonary inflammation.

The present methods for predicting, diagnosing, prognosticating and/or monitoring the diseases or conditions may be used in individuals who have not yet been diagnosed as having such (for example, preventative screening), or who have been diagnosed as having such, or who are suspected of having such (for example, display one or more characteristic symptoms), or who are at risk of developing such (for example, genetic predisposition; presence of one or more developmental, environmental or behavioural risk factors). The methods may also be used to detect various stages of progression or severity of the diseases or conditions. The methods may also be used to detect response of the diseases or conditions to prophylactic or therapeutic treatments or other interventions. The methods can furthermore be used to help the medical practitioner in deciding upon worsening, status-quo, partial recovery, or complete recovery of the patient from the diseases or conditions, resulting in either further treatment or observation or in discharge of the patient from medical care centre.

Any one of the herein described methods for predicting, diagnosing, prognosticating and/or monitoring the diseases or conditions may be employed for population screening (such as, e.g., screening in a general population or in a population stratified based on one or more criteria, e.g., age, gender, ancestry, occupation, presence or absence of risk factors of AHF, etc.). In any one the methods, the subject may form part of a patient population showing symptoms of dyspnea. In any one the methods, the subject may form part of a patient population showing symptoms or signs of an inflammatory condition such as sepsis, SIRS, or pulmonary inflammation.

Diabetes and hypertension represent major risk factors for developing renal dysfunction, more particularly (chronic) kidney failure. Hence, the present diagnosis, prediction, prognosis and/or monitoring methods may be preferably employed in such patients and patient populations, i.e., in subjects having or being at risk of having diabetes and/or hypertension (such as, e.g., in a screening setup).

The present methods enable the medical practitioner to monitor the disease progress by measuring the level of LTBP2 in a sample of the patient. For example, a decrease in LTBP2 level as compared to a prior LTBP2 level (e.g., at the time of the admission to ED) indicates the disease or condition in the subject is improving or has improved, while an increase of the LTBP2 level as compared to a prior LTBP2 level (e.g., at the time of the admission to ED) indicates the disease or condition in the subject has worsened or is worsening. Such worsening could possibly result in the recurrence of the disease or conditions.

In view of the present disclosure, also provided are:
the use of LTBP2 as a marker (biomarker);
the use of LTBP2 as a marker (biomarker) for any one disease or condition as taught herein;
the use of LTBP2 for diagnosis, prediction, prognosis and/or monitoring;
the use of LTBP2 for diagnosis, prediction, prognosis and/or monitoring of any one disease or condition as taught herein;
   particularly wherein said condition or disease may be chosen from increased mortality or risk of death of subjects having dyspnea and/or acute heart failure and/or renal dysfunction, increased risk of death of subjects presenting with one or more signs of an inflammatory condition such as sepsis, SIRS, or pulmonary inflammation.

In the present prediction, diagnosis, prognosis and/or monitoring methods the measurement of LTBP2 may also be combined with the assessment of one or more further biomarkers or clinical parameters relevant for the respective diseases and conditions.

In certain embodiments, the methods as taught herein may further comprise measuring the presence or absence and/or quantity of one or more other biomarkers useful for evaluating the risk of death within a year in the sample from the subject.

Hence, in certain embodiments, the method may comprise the steps of: (i) measuring the quantity of LTBP2 and the presence or absence and/or quantity of said one or more other biomarkers in the sample from the subject; (ii) establishing a subject profile of the quantity of LTBP2 and the presence or absence and/or quantity of said one or more other biomarkers using the measurements of (i); (iii) comparing said subject profile of (ii) to a reference profile of the quantity of LTBP2 and the presence or absence and/or quantity of said one or more other biomarkers, said reference profile representing a known risk of death such as a known risk of death within a year, for example within about 6 months, within about 5 months, within about 4 months, within about 3 months, within about 2 months, or within about one month, for a subject having an inflammatory condition; (iv) predicting an increased risk of death within a year, for example within about 6 months, within about 5 months, within about 4 months, within about 3 months, within about 2 months, or within about one month, for the subject if the quantity of LTBP2 measured in (ii) substantially corresponds to a reference value representing a subject having an inflammatory condition which will decease within a year, for example within about 6 months, within about 5 months, within about 4 months, within about 3 months, within about 2 months, or within about one month, or if the quantity of LTBP2 measured in (ii) is elevated compared with a reference value representing a subject having an inflammatory condition which will survive within a year, for example within about 6 months, within about 5 months, within about 4 months, within about 3 months, within about 2 months, or within about one month.

Consequently, in certain embodiments, the methods as taught herein, for instance the examination phase of the methods as taught herein, may further comprise measuring the presence or absence and/or quantity of one or more such other markers in the sample from the subject. In certain embodiments, the methods as taught herein may further comprise measuring the presence or absence and/or quantity of one or more other biomarkers useful for evaluating the risk of death within a year in the sample from the subject. In this respect, any known or yet unknown suitable marker could be used.

A reference throughout this specification to biomarkers “other than LTBP2” or “other biomarkers” generally encompasses such other biomarkers which are useful for the methods as disclosed herein. By means of example, biomarkers useful in evaluating the risk of death within a year, for example within about 6 months, within about 5 months, within about 4 months, within about 3 months, within about 2 months, or within about one month, for a subject presenting with one or more signs of an inflammatory condition include ST-2, galectin-3, midregional pro-adrenomedullin, creatinine (i.e., serum creatinine clearance), Cystatin C and neutrophil gelatinase-associated lipocalin (NGAL), beta-trace protein, kidney injury molecule 1 (KIM-1), interleukin-18 (IL-18), such as creatinine, Cystatin C and NGAL, beta-trace protein, KIM-1, IL-18, preferably ST-2, galectin-3, midregional pro-adrenomedullin, creatinine (i.e., serum creatinine clearance), Cystatin C. Further biomarkers useful in the present disclosure include inter alia B-type natriuretic peptide (BNP), pro-B-type natriuretic peptide (proBNP), amino terminal pro-B-type natriuretic peptide (NTproBNP) and C-reactive peptide, and fragments or precursors of any one thereof.

In certain embodiments, said other biomarker is chosen from the group consisting of ST-2, galectin-3, midregional pro-adrenomedullin, creatinine, Cystatin C, NGAL, beta-trace protein, KIM-1, IL-18, BNP, proBNP, NTproBNP and C-reactive peptide, and fragments or precursors of any one thereof. In certain preferred embodiments, said other biomarker is chosen from the group consisting of ST-2, galectin-3, midregional pro-adrenomedullin, creatinine, Cystatin C, BNP, proBNP, NTproBNP and C-reactive peptide, and fragments or precursors of any one thereof.

Hence, disclosed is a method for predicting, diagnosing and/or prognosticating the diseases or conditions as taught herein in a subject comprising the steps: (i) measuring the quantity of LTBP2 and the presence or absence and/or quantity of said one or more other biomarkers in the sample from the subject; (ii) using the measurements of (i) to establish a subject profile of the quantity of LTBP2 and the presence or absence and/or quantity of said one or more other biomarkers; (iii) comparing said subject profile of (ii) to a reference profile of the quantity of LTBP2 and the presence or absence and/or quantity of said one or more other biomarkers, said reference profile representing a known prediction, diagnosis and/or prognosis of the conditions, symptoms and/or parameter values according to the invention; (iv) finding a deviation or no deviation of the subject profile of (ii) from the reference profile; (v) attributing said finding of deviation or no deviation to a particular prediction, diagnosis and/or prognosis of the respective diseases or conditions in the subject.

Applying said method at two or more successive time points allows for monitoring the desired diseases or conditions.

The present methods may employ reference values for the quantity of LTBP2, which may be established according to known procedures previously employed for other biomarkers. Such reference values may be established either within (i.e., constituting a step of) or external to (i.e., not constituting a step of) the methods of the present invention as defined herein. Accordingly, any one of the methods taught herein may comprise a step of establishing a reference value for the quantity of LTBP2, said reference value representing either (a) a prediction or diagnosis of the absence of the diseases or as taught herein or a good prognosis thereof, or (b) a prediction or diagnosis of the diseases or conditions as taught herein or a poor prognosis thereof. Also, any one of the methods taught herein may comprise a step of establishing a reference value for the quantity of LTBP2, said reference value representing either (a) a prediction that the subject will survive in a given time interval such as within a year, for example within about 6 months, within about 5 months, within about 4 months, within about 3 months, within about 2 months, or within about one month, preferably within about one month, or a good prognosis of the subject, or (b) a prediction that the subject will decease in a given time interval such as within a year, for example within about 6 months, within about 5 months, within about 4 months, within about 3 months, within about 2 months, or within about one month, preferably within about one month, or a poor prognosis of the subject. In a preferred embodiment, the subject may be a subject having an inflammatory condition such as sepsis or SIRS or such as pulmonary inflammation.

A further aspect provides a method for establishing a reference value for the quantity of LTBP2, said reference value representing:

(a) a prediction or diagnosis of the absence of the diseases or conditions as taught herein or a good prognosis thereof, or
(b) a prediction or diagnosis of the diseases or conditions as taught herein or a poor prognosis thereof,
comprising:
(i) measuring the quantity of LTBP2 in:
(i a) one or more samples from one or more subjects not having the respective diseases or conditions or not being at risk of having such or having a good prognosis for such, or
(i b) one or more samples from one or more subjects having the respective diseases or conditions or being at risk of having such or having a poor prognosis for such, and
   (ii) storing the quantity of LTBP2
(ii a) as measured in (i a) as the reference value representing the prediction or diagnosis of the absence of the respective diseases or conditions or representing the good prognosis therefore, or
(ii b) as measured in (i b) as the reference value representing the prediction or diagnosis of the respective diseases or conditions or representing the poor prognosis therefore.

Also provided herein is a method for establishing a reference value for the quantity of LTBP2, said reference value representing:

(a) a prediction that the subject presenting with one or more signs of an inflammatory condition will survive in a given time interval such as within a year, for example within about 6 months, within about 5 months, within about 4 months, within about 3 months, within about 2 months, or within about one month, preferably within about one month, or a good prognosis thereof, or
(b) a prediction that the subject presenting with one or more signs of an inflammatory condition will decease in a given time interval such as within a year, for example within about 6 months, within about 5 months, within about 4 months, within about 3 months, within about 2 months, or within about one month, preferably within about one month, or a poor prognosis thereof,
said method may comprise:
(i) measuring the quantity of LTBP2 in:
(i a) one or more samples from one or more subjects having an inflammatory condition that will survive in a given time interval, or not being at risk of deceasing in a given time interval, or having a good prognosis for such, or
(i b) one or more samples from one or more subjects having an inflammatory condition that will decease in a given time interval, or being at risk of deceasing in a given time interval, or having a poor prognosis for such, and
   (ii) storing the quantity of LTBP2
(ii a) as measured in (i a) as the reference value representing the prediction of survival in a given time interval, or representing the good prognosis for the inflammatory condition, or
(ii b) as measured in (i b) as the reference value representing the prediction of non-survival or death in a given time interval, or representing the poor prognosis for the inflammatory condition.

The present methods may otherwise employ reference profiles for the quantity of LTBP2 and the presence or absence and/or quantity of one or more other biomarkers, which may be established according to known procedures previously employed for other biomarkers. Such reference profiles may be established either within (i.e., constituting a step of) or external to (i.e., not constituting a step of) the present methods. Accordingly, the methods taught herein may comprise a step of establishing a reference profile for the quantity of LTBP2 and the presence or absence and/or quantity of said one or more other biomarkers, said reference profile representing either (a) a prediction or diagnosis of the absence of the diseases or conditions as taught herein or a good prognosis therefore, or (b) a prediction or diagnosis of the diseases or conditions as taught herein or a poor prognosis therefore. Also, any one of the methods taught herein may comprise a step of establishing a reference value for the quantity of LTBP2 and the presence or absence and/or quantity of said one or more other biomarkers, said reference value representing either (a) a prediction that the subject will survive in a given time interval such as within a year, for example within about 6 months, within about 5 months, within about 4 months, within about 3 months, within about 2 months, or within about one month, preferably within about one month, or a good prognosis of the subject or (b) a prediction that the subject will decease in a given time interval such as within a year, for example within about 6 months, within about 5 months, within about 4 months, within about 3 months, within about 2 months, or within about one month, preferably within about one month, or a poor prognosis of the subject. In a preferred embodiment, the subject may be a subject having an inflammatory condition, preferably a systemic inflammatory condition such as sepsis or SIRS or such as pulmonary inflammation.

A further aspect provides a method for establishing a reference profile for the quantity of LTBP2 and the presence or absence and/or quantity of one or more other biomarkers useful for predicting, diagnosing, prognosticating and/or monitoring the diseases or conditions as taught herein, said reference profile representing:

(a) a prediction or diagnosis of the absence of the respective diseases or conditions or a good prognosis therefore, or
(b) a prediction or diagnosis of the respective diseases or conditions or a poor prognosis therefore,
comprising:
(i) measuring the quantity of LTBP2 and the presence or absence and/or quantity of said one or more other biomarkers in:
(i a) one or more samples from one or more subjects not having the respective diseases or conditions or not being at risk of having such or having a good prognosis for such; or
(i b) one or more samples from one or more subjects having the respective diseases or conditions or being at risk of having such or having a poor prognosis for such;
   (ii)
(ii a) using the measurements of (i a) to create a profile of the quantity of LTBP2 and the presence or absence and/or quantity of said one or more other biomarkers; or
(ii b) using the measurements of (i b) to create a profile of the quantity of LTBP2 and the presence or absence and/or quantity of said one or more other biomarkers;
   (iii)
(iii a) storing the profile of (ii a) as the reference profile representing the prediction or diagnosis of the absence of the respective diseases or conditions or representing the good prognosis therefore; or
(iii b) storing the profile of (ii b) as the reference profile representing the prediction or diagnosis of the respective diseases conditions or representing the poor prognosis therefore.

Also provided herein is a method for establishing a reference value for the quantity of LTBP2, said reference value representing:

(a) a prediction that the subject presenting with one or more signs of an inflammatory condition will survive in a given time interval such as within a year, for example within about 6 months, within about 5 months, within about 4 months, within about 3 months, within about 2 months, or within about one month, preferably within about one month, or a good prognosis thereof, or
(b) a prediction that the subject presenting with one or more signs of an inflammatory condition will decease in a given time interval such as within a year, for example within about 6 months, within about 5 months, within about 4 months, within about 3 months, within about 2 months, or within about one month, preferably within about one month, or a poor prognosis thereof,
said method may comprise:
(i) measuring the quantity of LTBP2 and the presence or absence and/or quantity of said one or more other biomarkers in:
(i a) one or more samples from one or more subjects having an inflammatory condition that will survive in a given time interval, or not being at risk of deceasing in a given time interval, or having a good prognosis for such, or
(i b) one or more samples from one or more subjects having an inflammatory condition that will decease in a given time interval, or being at risk of deceasing in a given time interval, or having a poor prognosis for such, and
   (ii)
(ii a) using the measurements of (i a) to create a profile of the quantity of LTBP2 and the presence or absence and/or quantity of said one or more other biomarkers; or
(ii b) using the measurements of (i b) to create a profile of the quantity of LTBP2 and the presence or absence and/or quantity of said one or more other biomarkers;
   (iii)
(iii a) storing the profile of (ii a) as the reference value representing the prediction of survival in a given time interval, or representing the good prognosis for the inflammatory condition, or
(iii b) storing the profile of (ii b) as the reference value representing the prediction of non-survival or death in a given time interval, or representing the poor prognosis for the inflammatory condition.

Further provided is a method for establishing a LTBP2 base-line or reference value in a subject, comprising: (i) measuring the quantity of LTBP2 in the sample from the subject at different time points wherein the subject is not suffering from the diseases or conditions as taught herein, and (ii) calculating the range or mean value of the subject, which is the LTBP2 base-line or reference value for said subject.

In certain embodiments, a method for establishing a LTBP2 base-line or reference value in a subject may comprising: (i) measuring the quantity of LTBP2 in the sample from the subject at different time points wherein the subject having dyspnea and/or acute heart failure and/or renal dysfunction or having an inflammatory condition, will not decease in a given time interval, and (ii) calculating the range or mean value of the subject, which is the LTBP2 base-line or reference value for said subject.

Preferably, the subject as intended in any one of the present methods may be human.

In certain embodiments, the quantity of LTBP2 and/or the presence or absence and/or quantity of the one or more other biomarkers may be measured by any suitable technique such as may be known in the art. For example, the quantity of LTBP2 and/or the presence or absence and/or quantity of the one or more other biomarkers may be measured using, respectively, a binding agent capable of specifically binding to LTBP2 and/or to fragments thereof, or a binding agent capable of specifically binding to said one or more other biomarkers. For example, the binding agent may be an antibody, aptamer, photoaptamer, Spiegelmer, protein, peptide, peptidomimetic or a small molecule, preferably the binding agent is an aptamer or antibody, more preferably, the binding agent is an aptamer.

In certain embodiments of the methods as taught herein, the quantity of LTBP2 and/or the presence or absence and/or quantity of the one or more other biomarkers may be measured using an immunoassay technology or a mass spectrometry analysis method or a chromatography method, or a combination of said methods.

In preferred embodiments of the methods as taught herein, the quantity of any one or more markers as taught herein, including LTBP2 and/or the presence or absence and/or quantity of the one or more other biomarkers, is measured using an immunoassay, e.g., an immunoassay employing antibody(ies) and/or aptamer(s), in preferred but non-limiting examples, using enzyme-linked immunosorbent assay (ELISA), radioimmunoassay (RIA), or ELISPOT technologies, preferably using ELISA.

In preferred embodiments of the methods as taught herein, the quantity of LTBP2 and/or the presence or absence and/or quantity of the one or more other biomarkers is measured using a binding agent capable of specifically binding to the respective markers, in preferred but non-limiting examples, using an aptamer, antibody, photoaptamer, Spiegelmer, protein, peptide, peptidomimetic, or a small molecule, preferably using an aptamer or antibody, more preferably using an aptamer.

Exemplary non-limiting specific antibodies for LTBP2 are commercially available, for instance, a goat polyclonal LTBP2 antibody (N-20) with catalogue number sc-18340 from Santa Cruz Biotechnology, Inc. (Santa Cruz, USA), or a rabbit polyclonal LTBP2 antibody with catalogue number ab121193 from Abcam (Cambridge, UK), or a rabbit polyclonal LTBP2 antibody with catalogue number 17708-1-AP from Acris Antibodies GmbH (Herford, Germany), or a Mouse anti Human LTBP2 5D7 antibody with catalogue number H00004053-M01 from Acris Antibodies GmbH (Herford, Germany).

Further disclosed is a kit for evaluating the risk of death within a year, for example within about 6 months, within about 5 months, within about 4 months, within about 3 months, within about 2 months, or within about one month, preferably within about one month, for a subject presenting with one or more signs of an inflammatory condition, the kit comprising (i) means for measuring the quantity of LTBP2 in a sample from the subject, and optionally and preferably (ii) a reference value of the quantity of LTBP2 or means for establishing said reference value, wherein said reference value represents a known risk of death such as a known risk of death within a year, for example within about 6 months, within about 5 months, within about 4 months, within about 3 months, within about 2 months, or within about one month, preferably within about one month, for a subject having an inflammatory condition. In certain preferred embodiments, said inflammatory condition may be sepsis or systemic inflammatory response syndrome (SIRS). In certain further preferred embodiments, said inflammatory condition may be pulmonary inflammation.

The kit thus allows one to: measure the quantity of LTBP2 in the sample from the subject by means (i); compare the quantity of LTBP2 measured by means (i) with the reference value of (ii) or established by means (ii); find a deviation or no deviation of the quantity of LTBP2 measured by means (i) from the reference value of (ii); and consequently attribute said finding of deviation or no deviation to a particular risk of death in the subject.

A further embodiment provides a kit for evaluating the risk of death within a year, for example within about 6 months, within about 5 months, within about 4 months, within about 3 months, within about 2 months, or within about one month, preferably within about one month, for a subject presenting with one or more signs of an inflammatory condition in a subject, the kit comprising (i) means for measuring the quantity of LTBP2 in a sample from the subject and (ii) means for measuring the presence or absence and/or quantity of one or more other biomarkers in the sample from the subject, and optionally and preferably (iii) means for establishing a subject profile of the quantity of LTBP2 and the presence or absence and/or quantity of said one or more other biomarkers, and optionally and preferably (iv) a reference profile of the quantity of LTBP2 and the presence or absence and/or quantity of said one or more other biomarkers, or means for establishing said reference profile, said reference profile representing a known risk of death such as a known risk of death within a year, for example within about 6 months, within about 5 months, within about 4 months, within about 3 months, within about 2 months, or within about one month, preferably within about one month, for a subject having an inflammatory condition. In certain preferred embodiments, said inflammatory condition may be sepsis or SIRS. In certain further preferred embodiments, said inflammatory condition may be pulmonary inflammation.

Such kit thus allows one to: measure the quantity of LTBP2 and the presence or absence and/or quantity of said one or more other biomarkers in the sample from the subject by respectively means (i) and (ii); establish (e.g., using means included in the kit or using suitable external means) a subject profile of the quantity of LTBP2 and the presence or absence and/or quantity of said one or more other biomarkers based on said measurements; compare the subject profile with the reference profile of (iv) or established by means (iv); find a deviation or no deviation of said subject profile from said reference profile; and consequently attribute said finding of deviation or no deviation to a particular risk of death in the subject.

The means for measuring the quantity of LTBP2 and/or the presence or absence and/or quantity of the one or more other biomarkers in the present kits may comprise, respectively, one or more binding agents capable of specifically binding to LTBP2 and/or to fragments thereof, and one or more binding agents capable of specifically binding to said one or more other biomarkers. For example, any one of said one or more binding agents may be an antibody, aptamer, photoaptamer, Spiegelmer, protein, peptide, peptidomimetic or a small molecule. For example, any one of said one or more binding agents may be advantageously immobilised on a solid phase or support. The means for measuring the quantity of LTBP2 and/or the presence or absence and/or quantity of the one or more other biomarkers in the present kits may employ an immunoassay technology or mass spectrometry analysis technology or chromatography technology, or a combination of said technologies.

Preferably, the present kits comprise one or more binding agents capable of specifically binding to said one or more markers as taught herein, including LTBP2, such as one or more aptamers, antibodies, photoaptamers, Spiegelmers, proteins, peptides, peptidomimetics or small molecules, preferably one or more aptamers or antibodies, more preferably one or more aptamers capable of specifically binding to said one or more markers as taught herein, including LTBP2. A binding agent may be advantageously immobilised on a solid phase or support.

The present kits may employ an immunoassay technology or mass spectrometry analysis technology or chromatography technology, or a combination of said technologies, preferably the present kits employ an immunoassay technology, in preferred but non-limiting examples, enzyme-linked immunosorbent assay (ELISA), radioimmunoassay (RIA), or ELISPOT technologies, preferably using ELISA. Hence, the means for measuring the quantity of marker(s) may be an immunoassay, e.g., an immunoassay employing antibody(ies) and/or aptamers, e.g., ELISA, RIA, or ELISPOT assay.

Disclosed is thus also a kit for evaluating the risk of death within a year, for example within about 6 months, within about 5 months, within about 4 months, within about 3 months, within about 2 months, or within about one month, preferably within about one month, for a subject presenting with one or more signs of an inflammatory condition, said kit comprising: (a) one or more binding agents capable of specifically binding to LTBP2 and/or to fragments thereof; (b) preferably, a known quantity or concentration of LTBP2 and/or a fragment thereof (e.g., for use as controls, standards and/or calibrators); (c) preferably, a reference value of the quantity of LTBP2, or means for establishing said reference value. Said components under (a) and/or (c) may be suitably labelled as taught elsewhere in this specification.

Also disclosed is a kit for evaluating the risk of death within a year, for example within about 6 months, within about 5 months, within about 4 months, within about 3 months, within about 2 months, or within about one month, preferably within about one month, for a subject presenting with one or more signs of an inflammatory condition, said kit comprising: (a) one or more binding agents capable of specifically binding to LTBP2 and/or to fragments thereof; (b) one or more binding agents capable of specifically binding to one or more other biomarkers; (c) preferably, a known quantity or concentration of LTBP2 and/or a fragment thereof and a known quantity or concentration of said one or more other biomarkers (e.g., for use as controls, standards and/or calibrators); (d) preferably, a reference profile of the quantity of LTBP2 and the presence or absence and/or quantity of said one or more other biomarkers, or means for establishing said reference profiles. Said components under (a), (b) and/or (c) may be suitably labelled as taught elsewhere in this specification.

Further disclosed is the use of the kit as described herein for evaluating the risk of death within a year, for example within about 6 months, within about 5 months, within about 4 months, within about 3 months, within about 2 months, or within about one month, preferably within about one month, for a subject presenting with one or more signs of an inflammatory condition as taught herein.

Also disclosed are reagents and tools useful for measuring LTBP2 and optionally the one or more other biomarkers concerned herein.

Hence, disclosed is a protein, polypeptide or peptide array or microarray comprising (a) LTBP2 and/or a fragment thereof, preferably a known quantity or concentration of said LTBP2 and/or fragment thereof; and (b) optionally and preferably, one or more other biomarkers, preferably a known quantity or concentration of said one or more other biomarkers.

Further provided is the use of any one protein, polypeptide or peptide array or microarray as described herein, for evaluating the risk of death within a year, for example within about 6 months, within about 5 months, within about 4 months, within about 3 months, within about 2 months, or within about one month, preferably within about one month, for a subject presenting with one or more signs of an inflammatory condition, preferably in a subject suspected or known to have an inflammatory condition, in a subject. In certain preferred embodiments, said inflammatory condition may be sepsis or SIRS. In certain further preferred embodiments, said inflammatory condition may be pulmonary inflammation.

Further disclosed is a protein, polypeptide or peptide array or microarray, in particular for performing the methods as taught herein, comprising one or more markers as taught herein, including LTBP2, preferably a known quantity or concentration of the one or more biomarkers.

Further disclosed is the use of any one protein, polypeptide or peptide array or microarray as described herein for evaluating the risk of death within a year, for example within about 6 months, within about 5 months, within about 4 months, within about 3 months, within about 2 months, or within about one month, preferably within about one month, for a subject presenting with one or more signs of an inflammatory condition, preferably in a subject suspected or known to have an inflammatory condition. In some preferred embodiments, said inflammatory condition may be sepsis or SIRS. In certain preferred embodiments, said inflammatory condition may be pulmonary injury.

Also disclosed is a binding agent array or microarray comprising: (a) one or more binding agents capable of specifically binding to LTBP2 and/or to fragments thereof, preferably a known quantity or concentration of said binding agents; and (b) optionally and preferably, one or more binding agents capable of specifically binding to one or more other biomarkers, preferably a known quantity or concentration of said binding agents.

Further provided is the use of any one binding agent array or microarray as described herein, for evaluating the risk of death within a year, for example within about 6 months, within about 5 months, within about 4 months, within about 3 months, within about 2 months, or within about one month, preferably within about one month, for a subject presenting with one or more signs of an inflammatory condition, preferably in a subject suspected or known to have an inflammatory condition. In particular, disclosed is the use of any one binding agent array or microarray as described herein comprising one or more binding agents capable of specifically binding to any one or more markers as taught herein, including LTBP2, in a sample from a subject, for performing any one of the methods as taught herein. Also intended herein is the use of any one binding agent array or microarray as described herein, wherein the binding agent array or microarray further comprises one or more binding agents useful for the prediction of mortality in a subject presenting with one or more signs of an inflammatory condition, preferably a known quantity or concentration of said binding agents.

Also disclosed are kits as taught here above configured as portable devices, such as, for example, bed-side devices, for use at home or in clinical settings, preferably in clinical settings.

A related aspect thus provides a portable testing device capable of measuring the quantity of LTBP2 in a sample from a subject comprising: (i) means for obtaining a sample from the subject, (ii) means for measuring the quantity of LTBP2 in said sample, and (iii) means for visualising the quantity of LTBP2 measured in the sample.

In an embodiment, the means of parts (ii) and (iii) may be the same, thus providing a portable testing device capable of measuring the quantity of LTBP2 in a sample from a subject comprising (i) means for obtaining a sample from the subject; and (ii) means for measuring the quantity of LTBP2 in said sample and visualising the quantity of LTBP2 measured in the sample.

In an embodiment, said visualising means is capable of indicating whether the quantity of LTBP2 in the sample is above or below a certain threshold level and/or whether the quantity of LTBP2 in the sample deviates or not from a reference value of the quantity of LTBP2, said reference value representing a known risk of death in a given time interval such as a known risk of death within a year, for example within about 6 months, within about 5 months, within about 4 months, within about 3 months, within about 2 months, or within about one month, in a subject having an inflammatory condition. Hence, the portable testing device may suitably also comprise said reference value or means for establishing the reference value.

In an embodiment, the threshold level is chosen such that the quantity of LTBP2 in the sample above said threshold level indicates that the subject has an increased risk of deceasing in a given time interval or indicates a poor prognosis for the subject, and the quantity of LTBP2 in the sample below said threshold level indicates that the subject does not have an increased risk of deceasing in a given time interval or indicates a good prognosis for the subject.

Hence, also disclosed herein are any one and all of the following:

(1) an agent that is able to modulate the level and/or the activity of LTBP2 for use as a medicament, preferably for use in the treatment of any one disease or condition as taught herein;
(2) use of an agent that is able to modulate the level and/or the activity of LTBP2 for the manufacture of a medicament for the treatment of any one disease or condition as taught herein; or use of an agent that is able to modulate the level and/or the activity of LTBP2 for the treatment of any one disease or condition as taught herein;
(3) a method for treating any one disease or condition as taught herein in a subject in need of such treatment, comprising administering to said subject a therapeutically or prophylactically effective amount of an agent that is able to modulate the level and/or the activity of LTBP2;
(4) The subject matter as set forth in any one of (1) to (3) above, wherein the agent is able to reduce or increase the level and/or the activity of LTBP2, preferably to reduce the level and/or the activity of LTBP2.
(5) The subject matter as set forth in any one of (1) to (4) above, wherein said agent is able to specifically bind to LTBP2.
(6) The subject matter as set forth in any one of (1) to (5) above, wherein said agent is an antibody or a fragment or derivative thereof; a polypeptide; a peptide; a peptidomimetic; an aptamer; a photoaptamer; a Spiegelmer; or a chemical substance, preferably an organic molecule, more preferably a small organic molecule.
(7) The subject matter as set forth in any one of (1) to (4) above, wherein the agent is able to reduce or inhibit the expression of LTBP2, preferably wherein said agent is an antisense agent; a ribozyme; or an agent capable of causing RNA interference.
(8) The subject matter as set forth in any one of (1) to (4) above, wherein said agent is able to reduce or inhibit the level and/or activity of LTBP2, preferably wherein said agent is a recombinant or isolated deletion construct of the LTBP2 polypeptide having a dominant negative activity over the native LTBP2.
(9) An assay to select, from a group of test agents, a candidate agent potentially useful in the treatment of any one disease or condition as taught herein, said assay comprising determining whether a tested agent can modulate, such as increase or reduce and preferably reduce, the level and/or activity of LTBP2.
(10) The assay as set forth in (9) above, further comprising use of the selected candidate agent for the preparation of a composition for administration to and monitoring the prophylactic and/or therapeutic effect thereof in a non-human animal model, preferably a non-human mammal model, of any one disease or condition as taught herein.
(11) The agent isolated by the assay as set forth in (10) above.
(12) A pharmaceutical composition or formulation comprising a prophylactically and/or therapeutically effective amount of one or more agents as set forth in any one of (1) to (8) or (10) above, or a pharmaceutically acceptable N-oxide form, addition salt, prodrug or solvate thereof, and further comprising one or more of pharmaceutically acceptable carriers.
(13) A method for producing the pharmaceutical composition or formulation as set forth in (12) above, comprising admixing said one or more agents with said one or more pharmaceutically acceptable carriers.

Said condition or disease as set forth in any one of (1) to (13) above may be particularly chosen from renal dysfunction, dyspnea associated with or caused by renal failure, increased mortality of subjects having dyspnea and/or acute heart failure and/or renal dysfunction, left ventricular hypertrophy, cardiac fibrosis, PE and PAP.

Also contemplated is thus a method (a screening assay) for selecting an agent capable of specifically binding to LTBP2 (e.g., gene or protein) comprising: (a) providing one or more, preferably a plurality of, test LTBP2-binding agents; (b) selecting from the test LTBP2-binding agents of (a) those which bind to LTBP2; and (c) counter-selecting (i.e., removing) from the test LTBP2-binding agents selected in (b) those which bind to any one or more other, unintended or undesired, targets.

Binding between test LTBP2-binding agents and LTBP2 may be advantageously tested by contacting (i.e., combining, exposing or incubating) said LTBP2 with the test LTBP2-binding agents under conditions generally conducive for such binding. For example and without limitation, binding between test LTBP2-binding agents and the LTBP2 may be suitably tested in vitro; or may be tested in host cells or host organisms comprising the LTBP2 and exposed to or configured to express the test LTBP2-binding agents.

Without limitation, the LTPB2-binding or LTBP2-modulating agents may be capable of binding LTBP2 or modulating the activity and/or level of the LTBP2 in vitro, in a cell, in an organ and/or in an organism.

In the screening assays as set forth in any one of (9) and (10) above, modulation of the activity and/or level of the LTBP2 by test LTBP2-modulating agents may be advantageously tested by contacting (i.e., combining, exposing or incubating) said LTBP2 (e.g., gene or protein) with the test LTBP2-modulating agents under conditions generally conducive for such modulation. By means of example and not limitation, where modulation of the activity and/or level of the LTBP2 results from binding of the test LTBP2-modulating agents to the LTBP2, said conditions may be generally conducive for such binding. For example and without limitation, modulation of the activity and/or level of the LTBP2 by test LTBP2-modulating agents may be suitably tested in vitro; or may be tested in host cells or host organisms comprising the LPBT2 and exposed to or configured to express the test LTBP2-modulating agents.

As well contemplated are:
LTBP2 for use as a medicament, preferably for use in the treatment of any one disease or condition as taught herein;
use of LTBP2 for the manufacture of a medicament for the treatment of any one disease or condition as taught herein;
use of LTBP2 for the treatment of any one disease or condition as taught herein;
a method for treating any one disease or condition as taught herein in a subject in need of such treatment, comprising administering to said subject a therapeutically or prophylactically effective amount of LTBP2;
   particularly wherein said condition or disease may be chosen from renal dysfunction, dyspnea associated with or caused by renal failure, increased mortality of subjects having dyspnea and/or acute heart failure and/or renal dysfunction, left ventricular hypertrophy, cardiac fibrosis, PE and PAP.

These and further aspects and preferred embodiments are described in the following sections and in the appended claims.

### BRIEF DESCRIPTION OF THE DRAWINGS

FIG. 1 illustrates sequences of full length LTBP2 (SEQ ID NO.1). The signal peptide is indicated in small caps. Also indicated is the selected MASSterclass quantified peptide (pept221—bold, italic, underlined/SEQ ID NO.2).

FIG. 2: (A) Box and whisker plots for LTBP2 at presentation in dyspneic patients as a function of survival at 1 year. (B) Rates of death at 1 year as a function of LTBP2 decile in all dyspneic patients.

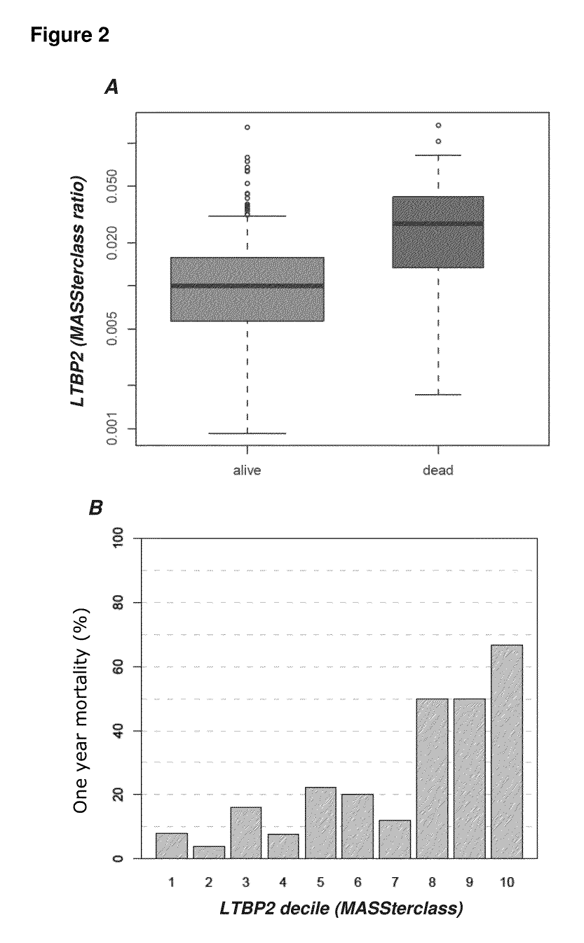

FIG. 3 illustrates box and whisker plots for LTBP2 levels at presentation as function of survival in dyspneic patients subdivided according to acute heart failure diagnosis (A) and kidney function (B). p-values shown are Wilcoxon rank sum p-values.

FIG. 4 shows receiver operating characteristic analysis comparing LTBP2 to cystatin C, CRP, BNP and NT-proBNP for predicting death at 1 year after presentation. Calculated median area under the curve (AUC) and 95% confidence intervals are: 0.77 (0.70-0.84) for LTBP2; 0.69 (0.62-0.77) for Cystatin C; 0.61 (0.55-0.68) for CRP; 0.72 (0.65-0.78) for BNP; 0.77 (0.70-0.83) for NTproBNP.

FIG. 5 Kaplan Meier survival plot illustrating the rates of death from presentation up 600 days of follow-up. The vertical grey line is the 1 year cut-off point. Among patients with high LTBP2 levels (above cut-off for maximal accuracy for predicting death at 1 year) a high mortality rate is observed. Log-rank p value is indicated.

FIG. 6 represents a box and whisker plot illustrating LTBP2 levels as measured by MASSterclass in survivor and non survivor patients presenting with signs of an inflammatory condition. Median levels are indicated.

FIGS. 7A and 7B represent box plot graphs illustrating LTBP2 normalized levels (FIG. 2A) and NTpro-BNP levels (pg/ml) (FIG. 2B) respectively in (A) 30 day survivors, (B) 30 day cardiac non-survivors and (C) 30 day pulmonary non-survivors. The p-value for survivors versus non-survivors because of pulmonary causes is <0.001.

FIGS. 8A and 8B represent box plot graphs illustrating LTBP2 normalized levels (FIG. 3A) and NTpro-BNP levels (pg/ml) (FIG. 3B) respectively in (A) one year survivors, (B) one year cardiac non-survivors and (C) one year pulmonary non-survivors. The p-value for survivors versus non-survivors because of pulmonary causes is <0.08.

FIG. 9 represents a bar chart illustrating the relationship between LTBP2 deciles and one-year all-cause mortality.

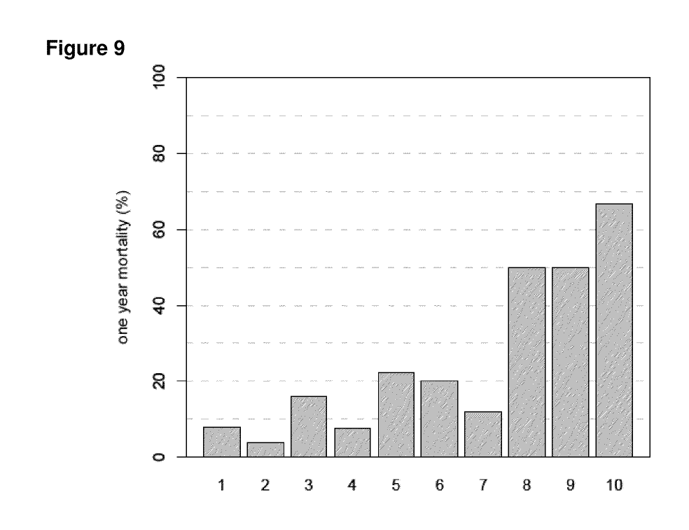

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The term “biomarker” is widespread in the art and may broadly denote a biological molecule and/or a detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition.

Reference herein to “disease(s) and/or condition(s) as taught herein” or a similar reference encompasses any such diseases and conditions as disclosed herein insofar consistent with the context of such a recitation, in particular but without limitation including renal dysfunction, dyspnea associated with or caused by renal failure, increased mortality of subjects having dyspnea and/or acute heart failure and/or renal dysfunction, increased mortality or risk of death of subjects presenting with one or more signs of an inflammatory condition.

Through extensive experimental testing, the inventors have found a method for evaluating the risk of death within a year for a subject presenting with one or more signs of an inflammatory condition such as sepsis or SIRS or such as pulmonary inflammation.

Sepsis may be characterized as mild sepsis, severe sepsis (sepsis with acute organ dysfunction), septic shock (sepsis with refractory arterial hypotension), organ failure, multiple organ dysfunction syndrome and death.

“Sepsis” can generally be defined as SIRS with a documented infection, such as for example a bacterial infection. Infection can be diagnosed by standard textbook criteria or, in case of uncertainty, by an infectious disease specialist. Bacteraemia is defined as sepsis where bacteria can be cultured from blood.

“SIRS” is an inflammatory response syndrome with no signs of infection. It can be characterized by the presence of at least two of the four following clinical criteria: fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCO₂ less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms.

“Mild sepsis” can be defined as the presence of sepsis without organ dysfunction.

“Severe sepsis” can be defined as the presence of sepsis and at least one of the following manifestations of organ hypoperfusion or dysfunction: hypoxemia, metabolic acidosis, oliguria, lactic acidosis, or an acute alteration in mental status without sedation.

“Septic shock” can be defined as the presence of sepsis accompanied by a sustained decrease in systolic blood pressure (90 mm Hg or less, or a drop of at least 40 mm Hg from baseline systolic blood pressure) despite fluid resuscitation, and the need for vasoactive amines to maintain adequate blood pressure.

Common sepsis-related definitions as may also be relied on here are further detailed in Levy M M et al., Crit. Care Med., 2003, vol. 31, 1250-56, or the definitions provided by the American College of Chest Physicians and the Society of Critical Care Medicine, Crit. Care Med., 1992, vol. 20: 864-874.

As many organisms may be the cause of sepsis, diagnosis often takes time and requires testing against panels of possible agents. Sepsis may also arise in many different circumstances and therefore sepsis may be further classified for example in: incarcerated sepsis which is an infection that is latent after the primary lesion has apparently healed but may be activated by a slight trauma; catheter sepsis which is sepsis occurring as a complication of intravenous catheterization; oral sepsis which is a disease condition in the mouth or adjacent parts which may affect the general health through the dissemination of toxins; puerperal sepsis which is infection of the female genital tract following childbirth, abortion, or miscarriage; or sepsis lenta, which is a condition produced by infection with a-hemolytic streptococci, characterized by a febrile illness with endocarditis.

The term “systemic inflammatory condition” as meant herein generally encompasses diseases and conditions comprising systemic inflammatory responses. The term particularly encompasses SIRS and sepsis and may more particularly refer to SIRS and/or sepsis.

Signs and symptoms of an inflammatory condition may encompass fever, muscle stiffness, joint pain and stiffness, headaches, and clinical data representative of an inflammatory condition such as the results of a patient chart, culture of micro-organisms, biochemical markers, treatment and response to treatment, etc.

For the purposes of this invention, the reference to a disease and/or condition is meant to include all stages of the progression of the disease and/or condition.

“Organ failure” may be defined as a condition where an organ does not perform its expected function. Organ failure relates to organ dysfunction to such a degree that normal homeostasis cannot be maintained without external clinical intervention. Examples of organ failure include without limitation renal failure, (acute) liver failure, heart failure, and respiratory failure.

“Multiple organ dysfunction syndrome” (MODS), “multiple organ failure” (MOF) or “multisystem organ failure” (MSOF) may be defined as altered organ function in an acutely ill patient requiring medical intervention to achieve homeostasis. It usually involves two or more organs or organ systems.

The terms “mortality” and “death” are well known per se and herein particularly relate to outcomes indicating that a subject may (e.g., with certain likelihood) or will die (i.e., permanent termination of the biological functions that sustain a living organism), particularly that the subject may or will die as a consequence of the disease or condition and/or that he/she will die within a given time period from sampling, such as several hours (e.g., between 1 and 24 hours or between 12 and 24 hours), several days (e.g., between 1 and 50 days or between 1 and 30 days), such as, for example within a month or within 4 weeks (28 days) or within a year, from sampling. The terms “die” or “decease” may be used interchangeably herein.

The terms “lung injury” or “pulmonary injury” may be used interchangeably herein and generally encompass damage to the lung(s) characterized by hypoxemia, non cardiogenic pulmonary edema, low lung compliance and/or widespread capillary leakage. Lung injury may be caused by any stimulus of local or systemic inflammation. Clinical features of lung injury comprise severe dyspnea, tachypnea, and resistant hypoxemia.

The terms “pulmonary inflammation” or “inflammation of the lung” may be used interchangeably herein and generally encompasses states, diseases and conditions in which the functioning of the lung or lung tissue is inadequate due to inflammation. The pulmonary inflammation may be caused by a septic event or an aseptic event or may be caused by inflammatory substances generated in another organ such as by inflammatory substances generated upon acute kidney injury or reperfusion injury of the heart. Signs and symptoms of pulmonary inflammation may include without limitation any one or more of cough; chest pain; fever; difficult breathing such as dyspnea; cyanosis or bluish skin; sharp chest pain; chest tightness; chills; sputum or mucus production; wheezing; weight loss; poor appetite and tiredness.

Dyspnea (dyspnoea or shortness of breath) is known per se and may particularly refer to a common and distressing symptom experienced by subjects as unpleasant or uncomfortable respiratory sensations that may be more particularly defined as a “subjective experience of breathing discomfort that consists of qualitatively distinct sensations that vary in intensity”. Dyspnea may be connected to a range of underlying pathologies.

The pulmonary inflammation caused by a septic event may be selected from one or more of pneumonia, bronchitis or chronic obstructive pulmonary disease (COPD).

The terms “pneumonia”, “bronchitis” and “chronic obstructive pulmonary disease” (COPD), as used herein, carry their respective art-established meanings. By means of further guidance, the term “pneumonia” generally refers to an inflammatory condition of the lung in particular affecting the microscopic air sacs or alveoli. Pneumonia may be caused by an infection by bacteria, viruses, fungi or parasites, or may be caused otherwise such as by autoimmune disease, chemicals or drugs. Pneumonia includes infectious pneumonia and noninfectious pneumonia or idiopathic interstitial pneumonia such as diffuse alveolar damage, organizing pneumonia, nonspecific interstitial pneumonia, lymphocytic interstitial pneumonia, desquamative interstitial pneumonia, respiratory bronchiolitis interstitial lung disease and usual interstitial pneumonia.

The term “bronchitis” generally refers to inflammation of the mucous membranes of the bronchi or airways that carry airflow from the trachea into the lungs. Bronchitis encompasses acute and chronic bronchitis. Acute bronchitis is characterized by the development of a cough, with or without the production of sputum or mucus that is expectorated (coughed up) from the respiratory tract. Acute bronchitis often occurs during the course of an acute viral illness such as the common cold or influenza. Chronic bronchitis, a type of chronic obstructive pulmonary disease, is characterized by the presence of a productive cough that lasts for three months or more per year for at least two years. Chronic bronchitis most often develops due to recurrent injury to the airways caused by inhaled irritants such as cigarette smoke or air pollution.

The term “chronic obstructive pulmonary disease” (COPD), also known as “chronic obstructive lung disease” (COLD), “chronic obstructive airway disease” (COAD), “chronic airflow limitation” (CAL) or “chronic obstructive respiratory disease” (CORD), is the co-occurrence of chronic bronchitis and emphysema.

Emphysema is know per se and may particularly refer to an enlargement of the air spaces distal to the terminal bronchioles, with destruction of their walls. The destruction of the air space walls reduces the surface area available for the exchange of oxygen and carbon dioxide during breathing and reduces the elasticity of the lung itself, which results in a loss of support for the airways that are embedded in the lung. These airways are more likely to collapse causing further limitation to airflow.

The pulmonary inflammation caused by an aseptic event may be selected from one or more of silicosis, ischemia, anaphylactic episode or lupus.

The term “silicosis”, also known as Potter's rot, is a form of occupational lung disease caused by inhalation of crystalline silica dust. Silicosis is typically marked by inflammation and scarring in forms of nodular lesions in the upper lobes of the lungs.

The terms “ischemia”, “ischaemia” or “ischemic stress” generally refer to a disease or condition characterized by a restriction in blood supply, i.e. a shortage of oxygen, glucose and other blood-borne nutrients, with resultant damage or dysfunction of tissue. Ischemia can be renal ischemia, myocardial ischemia, brain ischemia, mesenteric ischemia, ischemic colitis, ischemic stroke, limb ischemia or cutaneous ischemia. Ischemia can be chronic or acute.

The terms “anaphylactic episode” or “anaphylaxis” generally refer to a serious allergic reaction that is rapid in onset and may cause death. Anaphylaxis can result in a number of symptoms including throat swelling, an itchy rash, and low blood pressure.

The term “lupus”, also known as “systemic lupus erythematosus” (SLE), is an autoimmune disease (or autoimmune connective tissue disease) that can affect any part of the body. Lupus may refer to a Type III hypersensitivity reaction caused by antibody-immune complex formation. There is no one specific cause of SLE, however, SLE may be caused by a number of environmental triggers and by genetic susceptibility.

The pulmonary inflammation may be caused by inflammatory substances generated in another organ such as by inflammatory substances generated upon acute kidney injury or reperfusion injury of the heart or brain.

The inflammatory substances may be Proinflammatory cytokines, interferon gamma, IL-2, IL-10, granulocyte-macrophage colony-stimulating factor (GM-CSF), TGF-beta, IL 8 (CXCL1), IL-6, IL-18, macrophage inflammatory protein (MIP-)-2, monocyte chemoattractant protein (MCP)-1 are increased in kidney ischemia but also: IL-1beta, IL-1alfa, TNF-alfa are increased in cisplatin-induced AKI. Other markers include: Fractalkine (CX3CL1).

The complications related to pulmonary injury may encompass lung infarction, loss of functional lung tissue, emphysemia, lung fibrosis, atelectasis, pleuritis, pulmonary hypertension.

The term “lung fibrosis” or “pulmonary fibrosis”, also described as “scarring of the lung”, generally refers to the formation or development of excess fibrous connective tissue in the lungs.

Renal or kidney dysfunction, which may also be interchangeably known as renal or kidney failure or insufficiency, generally encompasses states, diseases and conditions in which the functioning of renal tissue is inadequate, particularly wherein kidney excretory function is compromised.

Signs and symptoms of renal dysfunction may include without limitation any one or more of increased levels of urea and/or nitrogen in the blood; lower than normal creatinine clearance and higher than normal creatinine levels in blood; lower than normal free water clearance; volume overload and swelling; abnormal acid levels; higher than normal levels of potassium, calcium and/or phosphate in blood; changes in urination (e.g., volume, osmolarity); microalbuminuria or macroalbuminuria; altered activity of kidney enzymes such as gamma glutamyl synthetase; fatigue; skin rash or itching; nausea; dyspnea; reduced kidney size; haematuria and anaemia.

Conventionally, renal dysfunction is deemed as comprising major classes denoted as acute renal or kidney failure (acute renal or kidney disease or injury, e.g., acute kidney injury or “AKI”) or chronic renal or kidney failure (chronic renal or kidney disease). Whereas progression is typically fast (e.g., days to weeks) in acute renal failure, renal failure may be traditionally regarded as chronic if it persists for at least 3 months and its progression may take in the range of years.

Acute renal dysfunction or failure may be staged (classified, graded) into 5 distinct stages using the “RIFLE” (Risk, Injury, Failure, Loss, end-stage renal disease) staging system as set out here below (based on Lameire et al. 2005, Lancet 365: 417-430):

Stage GFR (based on serum creatinine) criteria Urine output criteria
GFR=glomerular filtration rate
   “Risk” Serum creatinine increased 1.5 times <0.5 mL/kg/h for 6 h
   “Injury” Serum creatinine increased 2.0 times <0.5 mL/kg/h for 12 h
   “Failure” Serum creatinine increased 3.0 times, <0.3 mL/kg/h for 24 h or creatinine >355 mM/L when there or anuria for 12 h
was an acute rise of >44 mM/L
   “Loss” Persistent acute renal failure >4 weeks ----
   “End-stage” End-stage renal disease >3 months ---

Chronic renal dysfunction or failure may be staged (classified, graded) based on GFR as set out here below (based on Levey et al. 2005, Kidney Int 67: 2089-2100):

Stage 1: GFR≧90 mL/min (normal or elevated GFR)
Stage 2: GFR=60-89 mL/min (mild GFR reduction)
Stage 3: GFR=30-59 mL/min (moderate GFR reduction)
Stage 4: GFR=15-29 mL/min (severe GFR reduction)
Stage 5: GFR<15 mL/min (renal failure)

Other staging methods for renal failure resulting in similar or comparable classifications of different stages of renal failure may be used herein.

The present diagnosis, prediction, prognosis and/or monitoring methods may allow to determine that a subject has or is at risk of having acute or chronic renal failure, such as in particular determine any one of the above-described or comparable stages of acute or chronic renal failure in the subject, and/or may allow to discriminate between said stages in the subject.

The causes of acute renal deterioration may be pre-renal, post-renal and/or intra-renal. Pre-renal causes include lack of sufficient blood supply to the kidneys (i.e., renal hypoperfusion), which in turn may be caused by inter alia haemorrhage, massive blood loss, congestive heart failure, decompensated liver cirrhosis (liver cirrhosis with complications such as bleedings, ascites), damaged kidney blood vessels, sepsis or systemic inflammation due to infection. Post-renal causes include obstructions of urine collection systems or extra-renal drainage (i.e., obstructive uropathy), which in turn may be caused by inter alia medication interfering with normal bladder emptying, prostate diseases, kidney stones, abdominal malignancy (such as ovarian cancer or colorectal cancer), or obstructed urinary catheter. Intra-renal causes include renal tissue-destroying conditions, such as vasculitis, malignant hypertension, acute glomerulonephritis, acute interstitial nephritis and acute tubular necrosis. They can be caused without limitation by ischemic events (such as, e.g., haemoglobinuria, myoglobinuria and myoloma) or by nephrotoxic substances (such as, e.g., antibiotics, radio contrast agents, uric acid, oxalate and drug induced renal toxicity). Subjects having or being at risk of having the above states, conditions or diseases may have or may be at risk of developing acute renal failure. Hence, the present diagnosis, prediction, prognosis and/or monitoring methods may be preferably employed in such patients.

Causes of chronic renal deterioration may include inter alia vascular diseases, such as, e.g., bilateral renal artery stenosis, ischemic nephropathy, haemolytic-uremic syndrome and vasculitis, and further focal segmental nephrosclerosis, glomerulosclerosis, glomerulonephritis, IgA nephritis, diabetic nephropathy, lupus nephritis, polycystic kidney disease, chronic tubulointerstitial nephritis (e.g., drug and/or toxin-induced), renal fibrosis, nephronophthisis, kidney stones, and prostate diseases. Subjects having or being at risk of having the above states, conditions or diseases may have or may be at risk of developing chronic renal failure. Hence, the present diagnosis, prediction, prognosis and/or monitoring methods may be preferably employed in such patients.

The terms “heart failure”, “acute heart failure (AHF)” and “chronic heart failure (CHF)” as used herein carry their respective art-established meanings. By means of further guidance, the term “heart failure” as used herein broadly refers to pathological conditions characterised by an impaired diastolic or systolic blood flow rate and thus insufficient blood flow from the ventricle to peripheral organs.

“Acute heart failure” or also termed “acute decompensated heart failure” may be defined as the rapid onset of symptoms and signs secondary to abnormal cardiac function, resulting in the need for urgent therapy. AHF can present itself acute de novo (new onset of acute heart failure in a patient without previously known cardiac dysfunction) or as acute decompensation of CHF.

The cardiac dysfunction may be related to systolic or diastolic dysfunction, to abnormalities in cardiac rhythm, or to preload and afterload mismatch. It is often life threatening and requires urgent treatment. According to established classification, AHF includes several distinct clinical conditions of presenting patients: (I) acute decompensated congestive heart failure, (II) AHF with hypertension/hypertensive crisis, (III) AHF with pulmonary oedema, (IVa) cardiogenic shock/low output syndrome, (IVb) severe cardiogenic shock, (V) high output failure, and (VI) right-sided acute heart failure. For detailed clinical description, classification and diagnosis of AHF, and for summary of further AHF classification systems including the Killip classification, the Forrester classification and the ‘clinical severity’ classification, refer inter alia to Nieminen et al. 2005 (“Executive summary of the guidelines on the diagnosis and treatment of acute heart failure: the Task Force on Acute Heart Failure of the European Society of Cardiology”. Eur Heart J 26: 384-416) and references therein.

The term “chronic heart failure” (CHF) generally refers to a case of heart failure that progresses so slowly that various compensatory mechanisms work to bring the disease into equilibrium. Common clinical symptoms of CHF include inter alia any one or more of breathlessness, diminishing exercise capacity, fatigue, lethargy and peripheral oedema. Other less common symptoms include any one or more of palpitations, memory or sleep disturbance and confusion, and usually co-occur with one or more of the above recited common symptoms.

The terms “predicting” or “prediction”, “diagnosing” or “diagnosis” and “prognosticating” or “prognosis” are commonplace and well-understood in medical and clinical practice. It shall be understood that the phrase “a method for predicting, diagnosing and/or prognosticating” a given disease or condition may also be interchanged with phrases such as “a method for prediction, diagnosis and/or prognosis” of said disease or condition or “a method for making (or determining or establishing) a prediction, diagnosis and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The terms “predicting mortality” and “evaluating the risk of death” may be used interchangeably herein.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery.

A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period.

A poor prognosis of the diseases or conditions as taught herein may generally encompass anticipation of a substandard recovery and/or unsatisfactorily slow recovery, or to substantially no recovery or even further worsening of such.

The term “subject” or “patient” as used herein typically denotes humans, but may also encompass reference to non-human animals, preferably warm-blooded animals, more preferably mammals, such as, e.g., non-human primates, rodents, canines, felines, equines, ovines, porcines, and the like. Subjects typically include both male and female genders.

In certain embodiments of the present methods, the subject is a critically ill patient. The term “critically ill subject” may be used interchangeably herein with the recitations “subject with a condition requiring critical care”, “subject with a critical illness” or “subject with a critical care condition”.

The terms “critically ill”, “critical illness”, “condition which requires critical care”, or “critical care condition” are used interchangeably herein and generally refer to a condition which is life threatening to the sufferer and may thus result in death within a relatively short period of time such as within hours or days. Such conditions require critical care (e.g. monitoring and treatment) that generally involves close, constant attention by a team of specially trained health professionals. Such care usually takes place in an intensive care unit (ICU), emergency department (ED) or trauma centre. However, care might take place in any appropriate unit which has a similar or equivalent structure and capability as an ICU, ED or trauma centre. Thus, preferred critical conditions for application of the methods of the present invention are conditions requiring admittance to an ICU, ED or a setting which has a similar or equivalent structure and capability such as a trauma centre and preferred patients are ICU patients, ED patients or trauma centre patients.

Such critical care conditions include complications from surgery, life threatening accidents or other life threatening physical trauma or stress; medical shock i.e., a condition when insufficient blood flow reaches body tissues; infections e.g., bacterial, fungal or viral infections; systemic inflammatory response syndrome (SIRS); sepsis; severe sepsis i.e. sepsis with organ dysfunction; septic shock i.e., sepsis with acute circulatory failure; Acute Respiratory Distress Syndrome (ARDS) defined by pulmonary and systemic inflammation and pulmonary tissue injury (including endothelial and/or epithelial tissue) injury that result in alveolar filling and respiratory failure (Bajwa et al., Crit. Care Med., 2007, 35, 2484-2490); severe pneumonia; respiratory failure particularly acute respiratory failure; respiratory distress; severe chronic obstructive pulmonary disease (COPD); subarachnoidal hemorrhage (SAH); (severe) stroke; asphyxia; neurological conditions; organ dysfunction; single or multiple organ failure (MOF); poisoning and intoxication; severe allergic reactions and anaphylaxis; acute gastrointestinal and abdominal conditions resulting in SIRS; burn injury; acute cerebral hemorrhage or infarction; and any condition for which the patient requires assisted (e.g. mechanical) ventilation. It should be noted that, by their very nature, such conditions which require critical care are serious, severe, life-threatening forms of illness.

In certain embodiments, the present methods may be particularly applied to subjects known or suspected to have an inflammatory condition as defined herein or to subjects having an inflammatory condition as defined herein.

In certain preferred embodiments, the present methods may be particularly applied to subjects known or suspected to have sepsis or SIRS or to subjects having sepsis or SIRS.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. Preferred samples may include ones comprising LTBP2 protein in detectable quantities. In preferred embodiments, the sample may be whole blood or a fractional component thereof such as, e.g., plasma, serum, or a cell pellet. In preferred embodiments, the sample is blood, serum, plasma or urine. Preferably the sample is readily obtainable by minimally invasive methods, allowing to remove or isolate said sample from the subject. Samples may also include tissue samples and biopsies, tissue homogenates and the like. Preferably, the sample used to detect LTBP2 levels is blood plasma. Also preferably, the sample used to detect LTBP2 levels is urine. The term “plasma” defines the colorless watery fluid of the blood that contains no cells, but in which the blood cells (erythrocytes, leukocytes, thrombocytes, etc.) are suspended, containing nutrients, sugars, proteins, minerals, enzymes, etc.

A molecule or analyte such as a protein, polypeptide or peptide, or a group of two or more molecules or analytes such as two or more proteins, polypeptides or peptides, is “measured” in a sample when the presence or absence and/or quantity of said molecule or analyte or of said group of molecules or analytes is detected or determined in the sample, preferably substantially to the exclusion of other molecules and analytes.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

As used herein, the term “LTBP2” corresponds to the protein commonly known as latent transforming growth factor beta binding protein 2 (LTBP2), also known as GLC3D, LTBP3, MSTP031, C14orf141, i.e. the proteins and polypeptides commonly known under these designations in the art. The terms encompass such proteins and polypeptides of any organism where found, and particularly of animals, preferably vertebrates, more preferably mammals, including humans and non-human mammals, even more preferably of humans. The terms particularly encompass such proteins and polypeptides with a native sequence, i.e., ones of which the primary sequence is the same as that of LTBP2 found in or derived from nature. A skilled person understands that native sequences of LTBP2 may differ between different species due to genetic divergence between such species. Moreover, the native sequences of LTBP2 may differ between or within different individuals of the same species due to normal genetic diversity (variation) within a given species. Also, the native sequences of LTBP2 may differ between or even within different individuals of the same species due to post-transcriptional or post-translational modifications. Accordingly, all LTBP2 sequences found in or derived from nature are considered “native”. The terms encompass LTBP2 proteins and polypeptides when forming a part of a living organism, organ, tissue or cell, when forming a part of a biological sample, as well as when at least partly isolated from such sources. The terms also encompass proteins and polypeptides when produced by recombinant or synthetic means.

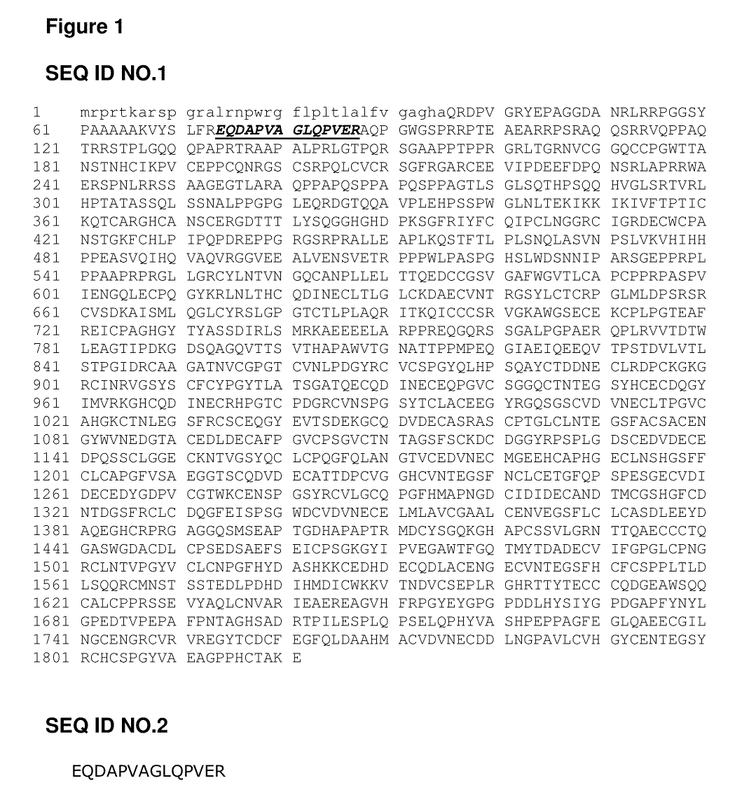

Exemplary LTBP2 includes, without limitation, human LTBP2 having primary amino acid sequence as annotated under NCBI Genbank accession number NP_{—}000419 (sequence version 1) as reproduced in FIG. 1 (SEQ ID NO: 1). A skilled person can also appreciate that said sequences are of precursor of LTBP2 and may include parts which are processed away from mature LTBP2. For example, in FIG. 1, an LTBP2 signal peptide is indicated in small caps in the amino acid sequence.

In an embodiment the circulating LTBP2, e.g., secreted form circulating in the blood plasma, may be detected, as opposed to the cell-bound or cell-confined LTBP2 protein.

The reference herein to LTBP2 may also encompass fragments of LTBP2. Hence, the reference herein to measuring LTBP2, or to measuring the quantity of LTBP2, may encompass measuring the LTBP2 protein or polypeptide, such as, e.g., measuring the mature and/or the processed soluble/secreted form (e.g. plasma circulating form) of LTBP2 and/or measuring one or more fragments thereof. For example, LTBP2 and/or one or more fragments thereof may be measured collectively, such that the measured quantity corresponds to the sum amounts of the collectively measured species. In another example, LTBP2 and/or one or more fragments thereof may be measured each individually. Preferably, said fragment of LTBP2 is a plasma circulating form of LTBP2. The expression “plasma circulating form of LTBP2” or shortly “circulating form” encompasses all LTBP2 proteins or fragments thereof that circulate in the plasma, i.e., are not cell- or membrane-bound. Without wanting to be bound by any theory, such circulating forms can be derived from the full-length LTBP2 protein through natural processing, or can be resulting from known degradation processes occurring in said sample. In certain situations, the circulating form can also be the full-length LTBP2 protein, which is found to be circulating in the plasma. Said “circulating form” can thus be any LTBP2 protein or any processed soluble form of LTBP2 or fragments of either one, that is circulating in the sample, i.e. which is not bound to a cell- or membrane fraction of said sample.

As used herein, the terms “pro-B-type natriuretic peptide” (also abbreviated as “proBNP”) and “amino terminal pro-B-type natriuretic peptide” (also abbreviated as “NTproBNP”) and “B-type natriuretic peptide” (also abbreviated as “BNP”) refer to peptides commonly known under these designations in the art. As further explanation and without limitation, in vivo proBNP, NTproBNP and BNP derive from natriuretic peptide precursor B preproprotein (preproBNP). In particular, proBNP peptide corresponds to the portion of preproBNP after removal of the N-terminal secretion signal (leader) sequence from preproBNP. NTproBNP corresponds to the N-terminal portion and BNP corresponds to the C-terminal portion of the proBNP peptide subsequent to cleavage of the latter C-terminally adjacent to amino acid 76 of proBNP.

The term “Cystatin C”, also known as ARMD11; MGC117328, Cystatin-3 (CST3), refers to peptides commonly known under these designations in the art, as exemplarily annotated under Genbank accession number NP_{—}000090 (sequence version 1).

As used herein, “neutrophil gelatinase-associated lipocalin” or “NGAL”, also known as oncogenic lipocalin 24P3, uterocalin or lipocalin 2 (LCN2), refers to peptides commonly known under these designations in the art, as exemplarily annotated under Genbank accession number NP_{—}005555 (sequence version 2).

The term “C-reactive protein”, also known as CRP or PTX1, refers to peptides commonly known under these designations in the art, as exemplarily annotated under Genbank accession number NP_{—}000558 (sequence version 2).

The term “beta-trace protein”, also known as inter alia prostaglandin-H2 D-isomerase, prostaglandin-D2 synthase, cerebrin-28 and PTGDS, refers to peptides commonly known under these designations in the art, as exemplarily annotated under Genbank accession number NP_{—}000945 (sequence version 3).

The term “kidney injury molecule 1” or KIM-1 refers to peptides commonly known under these designations in the art, as exemplarily disclosed in Ichimura et al. 2004 (Am J Physiol Renal Physiol 286(3): F552-63) and Ichimura et al. 1998 (J Biol Chem 273: 4135-4142).

The term “interleukin-18” refers to peptides commonly known under this designation in the art, as exemplarily annotated under Genbank accession number NP_{—}001553 (sequence version 1).

Unless otherwise apparent from the context, reference herein to any protein, polypeptide or peptide encompasses such from any organism where found, and particularly preferably from animals, preferably vertebrates, more preferably mammals, including humans and non-human mammals, even more preferably from humans.

Further, unless otherwise apparent from the context, reference herein to any protein, polypeptide or peptide and fragments thereof may generally also encompass modified forms of said protein, polypeptide or peptide and fragments such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

In an embodiment, LTBP2 and fragments thereof, or other biomarkers as employed herein and fragments thereof, may be human, i.e., their primary sequence may be the same as a corresponding primary sequence of or present in a naturally occurring human peptides, polypeptides or proteins. Hence, the qualifier “human” in this connection relates to the primary sequence of the respective proteins, polypeptides, peptides or fragments, rather than to their origin or source. For example, such proteins, polypeptides, peptides or fragments may be present in or isolated from samples of human subjects or may be obtained by other means (e.g., by recombinant expression, cell-free translation or non-biological peptide synthesis).

The term “fragment” of a protein, polypeptide or peptide generally refers to N-terminally and/or C-terminally deleted or truncated forms of said protein, polypeptide or peptide. The term encompasses fragments arising by any mechanism, such as, without limitation, by alternative translation, exo- and/or endo-proteolysis and/or degradation of said protein or polypeptide, such as, for example, in vivo or in vitro, such as, for example, by physical, chemical and/or enzymatic proteolysis. Without limitation, a fragment of a protein, polypeptide or peptide may represent at least about 5%, or at least about 10%, e.g., ≧20%, ≧30% or ≧40%, such as ≧50%, e.g., ≧60%, ≧70% or ≧80%, or even ≧90% or ≧95% of the amino acid sequence of said protein, polypeptide or peptide.

For example, a fragment may include a sequence of ≧5 consecutive amino acids, or ≧10 consecutive amino acids, or ≧20 consecutive amino acids, or ≧30 consecutive amino acids, e.g., ≧40 consecutive amino acids, such as for example ≧50 consecutive amino acids, e.g., ≧60, ≧70, ≧80, ≧90, ≧100, ≧200, ≧300, ≧400, ≧500 or ≧600 consecutive amino acids of the corresponding full length protein.

In an embodiment, a fragment may be N-terminally and/or C-terminally truncated by between 1 and about 20 amino acids, such as, e.g., by between 1 and about 15 amino acids, or by between 1 and about 10 amino acids, or by between 1 and about 5 amino acids, compared to the corresponding mature, full-length protein or its soluble or plasma circulating form. By means of example, proBNP, NTproBNP and BNP fragments useful as biomarkers are disclosed in WO 2004/094460.

In an embodiment, fragments of a given protein, polypeptide or peptide may be achieved by in vitro proteolysis of said protein, polypeptide or peptide to obtain advantageously detectable peptide(s) from a sample. For example, such proteolysis may be effected by suitable physical, chemical and/or enzymatic agents, e.g., proteinases, preferably endoproteinases, i.e., protease cleaving internally within a protein, polypeptide or peptide chain. A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases. Exemplary non-limiting endoproteinases include trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). Further known or yet to be identified enzymes may be used; a skilled person can choose suitable protease(s) on the basis of their cleavage specificity and frequency to achieve desired peptide forms. Preferably, the proteolysis may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, trypsin in solution, trypsin immobilised to a solid support, etc. Trypsin is particularly useful, inter alia due to high specificity and efficiency of cleavage.

The invention also contemplates the use of any trypsin-like protease, i.e., with a similar specificity to that of trypsin. Otherwise, chemical reagents may be used for proteolysis. For example, CNBr can cleave at Met; BNPS-skatole can cleave at Trp. The conditions for treatment, e.g., protein concentration, enzyme or chemical reagent concentration, pH, buffer, temperature, time, can be determined by the skilled person depending on the enzyme or chemical reagent employed.

Also provided is thus an isolated fragment of LTBP2 as defined here above. Such fragments may give useful information about the presence and quantity of LTBP2 in biological samples, whereby the detection of said fragments is of interest. Hence, the herein disclosed fragments of LTBP2 are useful biomarkers. A preferred LTBP2 fragment may comprise, consist essentially of or consist of the sequence as set forth in SEQ ID NO: 2.

The term “isolated” with reference to a particular component (such as for instance, a protein, polypeptide, peptide or fragment thereof) generally denotes that such component exists in separation from—for example, has been separated from or prepared in separation from—one or more other components of its natural environment. For instance, an isolated human or animal protein, polypeptide, peptide or fragment exists in separation from a human or animal body where it occurs naturally.

The term “isolated” as used herein may preferably also encompass the qualifier “purified”. As used herein, the term “purified” with reference to protein(s), polypeptide(s), peptide(s) and/or fragment(s) thereof does not require absolute purity. Instead, it denotes that such protein(s), polypeptide(s), peptide(s) and/or fragment(s) is (are) in a discrete environment in which their abundance (conveniently expressed in terms of mass or weight or concentration) relative to other proteins is greater than in a biological sample. A discrete environment denotes a single medium, such as for example a single solution, gel, precipitate, lyophilisate, etc. Purified peptides, polypeptides or fragments may be obtained by known methods including, for example, laboratory or recombinant synthesis, chromatography, preparative electrophoresis, centrifugation, precipitation, affinity purification, etc.

Purified protein(s), polypeptide(s), peptide(s) and/or fragment(s) may preferably constitute by weight ≧10%, more preferably ≧50%, such as ≧60%, yet more preferably ≧70%, such as ≧80%, and still more preferably ≧90%, such as ≧95%, ≧96%, ≧97%, ≧98%, ≧99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of peptides or polypeptides may be determined by SDS-PAGE under reducing or non-reducing conditions using Coomassie blue or, preferably, silver stain.

Further disclosed are isolated LTBP2 or fragments thereof as taught herein comprising a detectable label. This facilitates ready detection of such fragments. The term “label” as used throughout this specification refers to any atom, molecule, moiety or biomolecule that can be used to provide a detectable and preferably quantifiable read-out or property, and that can be attached to or made part of an entity of interest, such as a peptide or polypeptide or a specific-binding agent. Labels may be suitably detectable by mass spectrometric, spectroscopic, optical, colorimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish phosphatise or alkaline phosphatise as commonly used in immunoassays); binding moieties such as biotin-streptavidin; haptens such as digoxigenin; luminogenic, phosphorescent or fluorogenic moieties; mass tags; and fluorescent dyes alone or in combination with moieties that can suppress or shift emission spectra by fluorescence resonance energy transfer (FRET).

For example, the label may be a mass-altering label. Preferably, a mass-altering label may involve the presence of a distinct stable isotope in one or more amino acids of the peptide vis-à-vis its corresponding non-labelled peptide. Mass-labelled peptides are particularly useful as positive controls, standards and calibrators in mass spectrometry applications. In particular, peptides including one or more distinct isotopes are chemically alike, separate chromatographically and electrophoretically in the same manner and also ionise and fragment in the same way. However, in a suitable mass analyser such peptides and optionally select fragmentation ions thereof will display distinguishable m/z ratios and can thus be discriminated. Examples of pairs of distinguishable stable isotopes include H and D, ¹²C and ¹³C, ¹⁴N and ¹⁵N or ¹⁶O and ¹⁸O. Usually, peptides and proteins of biological samples analysed in the present invention may substantially only contain common isotopes having high prevalence in nature, such as for example H, ¹²C, ¹⁴N and ¹⁶O. In such case, the mass-labelled peptide may be labelled with one or more uncommon isotopes having low prevalence in nature, such as for instance D, ¹³C, ¹⁵N and/or ¹⁸O. It is also conceivable that in cases where the peptides or proteins of a biological sample would include one or more uncommon isotopes, the mass-labelled peptide may comprise the respective common isotope(s).

Isotopically-labelled synthetic peptides may be obtained inter alia by synthesising or recombinantly producing such peptides using one or more isotopically-labelled amino acid substrates, or by chemically or enzymatically modifying unlabelled peptides to introduce thereto one or more distinct isotopes. By means of example and not limitation, D-labelled peptides may be synthesised or recombinantly produced in the presence of commercially available deuterated L-methionine CH₃—S—CD₂CD₂-CH(NH₂)—COOH or deuterated arginine H₂NC(═NH)—NH—(CD₂)₃-CD(NH₂)—COOH. It shall be appreciated that any amino acid of which deuterated or ¹⁵N- or ¹³C-containing forms exist may be considered for synthesis or recombinant production of labelled peptides. In another non-limiting example, a peptide may be treated with trypsin in H₂¹⁶O or H₂¹⁸O, leading to incorporation of two oxygens (¹⁶O or ¹⁸O, respectively) at the COOH-termini of said peptide (e.g., US 2006/105415).

Accordingly, also contemplated is the use of LTBP2 and isolated fragments thereof as taught herein, optionally comprising a detectable label, as (positive) controls, standards or calibators in qualitative or quantitative detection assays (measurement methods) of LTBP2, and particularly in such methods for predicting, diagnosing, prognosticating and/or monitoring the diseases or conditions as taught herein in subjects. The proteins, polypeptides or peptides may be supplied in any form, inter alia as precipitate, vacuum-dried, lyophilisate, in solution as liquid or frozen, or covalently or non-covalently immobilised on solid phase, such as for example, on solid chromatographic matrix or on glass or plastic or other suitable surfaces (e.g., as a part of peptide arrays and microarrays). The peptides may be readily prepared, for example, isolated from natural sources, or prepared recombinantly or synthetically.

Further disclosed are binding agents capable of specifically binding to any one or more of the isolated fragments of LTBP2 as taught herein. Also disclosed are binding agents capable of specifically binding to only one of isolated fragments of LTBP2 as taught herein. Binding agents as intended throughout this specification may include inter alia an antibody, aptamer, photoaptamer, Spiegelmer, protein, peptide, peptidomimetic or a small molecule.

A binding agent may be capable of binding both the plasma circulating form and the cell-bound or retained from of LTBP2. Preferably, a binding agent may be capable of specifically binding or detecting the plasma circulating form of LTBP2.

The term “specifically bind” as used throughout this specification means that an agent (denoted herein also as “specific-binding agent”) binds to one or more desired molecules or analytes, such as to one or more proteins, polypeptides or peptides of interest or fragments thereof substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to protein(s) polypeptide(s), peptide(s) and/or fragment(s) thereof of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, the agent may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

Specific binding agents as used throughout this specification may include inter alia an antibody, aptamer, photoaptamer, Spiegelmer, protein, peptide, peptidomimetic or a small molecule.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The term “aptamer” refers to single-stranded or double-stranded oligo-DNA, oligo-RNA or oligo-DNA/RNA or any analogue thereof, that can specifically bind to a target molecule such as a peptide. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “Spiegelmer” refers to an aptamer built using L-ribose. Spiegelmers are the enantiomers of natural oligonucleotides, which are made with D-ribose. Due to their L-nucleotides, Spiegelmers are highly resistant to degradation by nucleases. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Harwell 1995 (Trends Biotechnol 13: 132-134).

The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

Hence, also disclosed are methods for immunising animals, e.g., non-human animals such as laboratory or farm, animals using (i.e., using as the immunising antigen) the herein taught fragments of LTBP2, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Immune sera obtained or obtainable by immunisation as taught herein may be particularly useful for generating antibody reagents that specifically bind to one or more of the herein disclosed fragments of LTBP2.

Further disclosed are methods for selecting specific-binding agents which bind (a) one or more of the LTBP2 fragments taught herein, substantially to the exclusion of (b) LTBP2 and/or other fragments thereof. Conveniently, such methods may be based on subtracting or removing binding agents which cross-react or cross-bind the non-desired LTBP2 molecules under (b). Such subtraction may be readily performed as known in the art by a variety of affinity separation methods, such as affinity chromatography, affinity solid phase extraction, affinity magnetic extraction, etc.

Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of LTBP2 and/or fragments thereof and optionally of the one or more other biomarkers or fragments thereof in samples (any molecules or analytes of interest to be so-measured in samples, including LTBP2 and fragments thereof, may be herein below referred to collectively as biomarkers).

For example, such methods may include immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly an antibody, and the molecule(s) or analyte(s) of interest. Immunoassay technologies include without limitation direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary antibody to bind to and thereby quantify target antigen in a sample immobilised on a solid support such as a microwell plate. Indirect ELISA uses a non-labelled primary antibody which binds to the target antigen and a secondary labelled antibody that recognises and allows to quantify the antigen-bound primary antibody. In sandwich ELISA the target antigen is captured from a sample using an immobilised ‘capture’ antibody which binds to one antigenic site within the antigen, and subsequent to removal of non-bound analytes the so-captured antigen is detected using a ‘detection’ antibody which binds to another antigenic site within said antigen, where the detection antibody may be directly labelled or indirectly detectable as above. Competitive ELISA uses a labelled ‘competitor’ that may either be the primary antibody or the target antigen. In an example, non-labelled immobilised primary antibody is incubated with a sample, this reaction is allowed to reach equilibrium, and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the endpoint is typically colorimetric, chemiluminescent or fluorescent, magnetic, piezo electric, pyroelectric and other.

Radioimmunoassay (RIA) is a competition-based technique and involves mixing known quantities of radioactively-labelled (e.g., ¹²⁵I- or ¹³¹I-labelled) target antigen with antibody to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Generally, any mass spectrometric (MS) techniques that can obtain precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID). Detection and quantification of biomarkers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods described herein below.

Chromatography can also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

Chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The various aspects and embodiments taught herein may further rely on comparing the quantity of LTBP2 measured in samples with reference values of the quantity of LTBP2, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such different between values or profiles being compared. A comparison may include a visual inspection, an arithmetical or statistical comparison of measurements. Such statistical comparisons include, but are not limited to, applying a rule. If the values or biomarker profiles comprise at least one standard, the comparison to determine a difference in said values or biomarker profiles may also include measurements of these standards, such that measurements of the biomarker are correlated to measurements of the internal standards.

Reference values for the quantity of LTBP2 may be established according to known procedures previously employed for other biomarkers.

For example, a reference value of the quantity of LTBP2 for a particular prediction, diagnosis and/or prognosis of given disease or condition as taught herein may be established by determining the quantity of LTBP2 in sample(s) from one individual or from a population of individuals characterised by said particular prediction, diagnosis and/or prognosis of said disease or condition (i.e., for whom said prediction, diagnosis and/or prognosis of renal dysfunction holds true). Such population may comprise without limitation ≧2, ≧10, ≧100, or even several hundreds or more individuals.

Hence, by means of an illustrative example, reference values of the quantity of LTBP2 for the diagnoses of a given disease or condition as taught herein vs. no such disease or condition may be established by determining the quantity of LTBP2 in sample(s) from one individual or from a population of individuals diagnosed (e.g., based on other adequately conclusive means, such as, for example, clinical signs and symptoms, imaging, ECG, etc.) as, respectively, having or not having said disease or condition.

In an embodiment, reference value(s) as intended herein may convey absolute quantities of LTBP2. In another embodiment, the quantity of LTBP2 in a sample from a tested subject may be determined directly relative to the reference value (e.g., in terms of increase or decrease, or fold-increase or fold-decrease). Advantageously, this may allow to compare the quantity of LTBP2 in the sample from the subject with the reference value (in other words to measure the relative quantity of LTBP2 in the sample from the subject vis-à-vis the reference value) without the need to first determine the respective absolute quantities of LTBP2.

The expression level or presence of a biomarker in a sample of a patient may sometimes fluctuate, i.e. increase or decrease significantly without change (appearance of, worsening or improving of) symptoms. In such an event, the marker change precedes the change in symptoms and becomes a more sensitive measure than symptom change. Therapeutic intervention can be initiated earlier and be more effective than waiting for deteriorating symptoms. Early intervention at a more benign status may be carried out safely at home, which is a major improvement from treating seriously deteriorated patients in the emergency room.

Measuring the LTBP2 level of the same patient at different time points can in such a case thus enable the continuous monitoring of the status of the patient and can lead to prediction of worsening or improvement of the patient's condition with regard to a given disease or condition as taught herein. A home or clinical test kit or device as indicated herein can be used for this continuous monitoring. One or more reference values or ranges of LTBP2 levels linked to a certain disease state (e.g. renal dysfunction or no renal dysfunction) for such a test can e.g. be determined beforehand or during the monitoring process over a certain period of time in said subject. Alternatively, these reference values or ranges can be established through data sets of several patients with highly similar disease phenotypes, e.g. from healthy subjects or subjects not having the disease or condition of interest. A sudden deviation of the LTBP2 levels from said reference value or range can predict the worsening of the condition of the patient (e.g. at home or in the clinic) before the (often severe) symptoms actually can be felt or observed.

Also disclosed is thus a method or algorithm for determining a significant change in the level of the LTBP2 marker in a certain patient, which is indicative for change (worsening or improving) in clinical status. In addition, the invention allows establishing the diagnosis that the subject is recovering or has recovered from a given disease or condition as taught herein.

In an embodiment the present methods may include a step of establishing such reference value(s). In an embodiment, the present kits and devices may include means for establishing a reference value of the quantity of LTBP2 for a particular prediction, diagnosis and/or prognosis of a given disease or condition as taught herein. Such means may for example comprise one or more samples (e.g., separate or pooled samples) from one or more individuals characterised by said particular prediction, diagnosis and/or prognosis of said disease or condition.

The various aspects and embodiments taught herein may further entail finding a deviation or no deviation between the quantity of LTBP2 measured in a sample from a subject and a given reference value.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD, or ±1×SE or ±2×SE). Deviation may also refer to a value falling outside of a reference range defined by values in a given population (for example, outside of a range which comprises ≧40%, ≧50%, ≧60%, ≧70%, ≧75% or ≧80% or ≧85% or ≧90% or ≧95% or even ≧100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction, diagnosis and/or prognosis methods, e.g., sensitivity and/or specificity of at least 50%, or at least 60%, or at least 70%, or at least 80%, or at least 85%, or at least 90%, or at least 95%.

For example, in an embodiment, an elevated quantity of LTBP2 in the sample from the subject—preferably at least about 1.1-fold elevated, or at least about 1.2-fold elevated, more preferably at least about 1.3-fold elevated, even more preferably at least about 1.4-fold elevated, yet more preferably at least about 1.5-fold elevated, such as between about 1.1-fold and 3-fold elevated or between about 1.5-fold and 2-fold elevated—compared to a reference value representing the prediction or diagnosis of no given disease or condition as taught herein or representing a good prognosis for said disease or condition indicates that the subject has or is at risk of having said disease or condition or indicates a poor prognosis for the disease or condition in the subject.

When a deviation is found between the quantity of LTBP2 in a sample from a subject and a reference value representing a certain prediction, diagnosis and/or prognosis of a given disease or condition as taught herein, said deviation is indicative of or may be attributed to the conclusion that the prediction, diagnosis and/or prognosis of said disease or condition in said subject is different from that represented by the reference value.

When no deviation is found between the quantity of LTBP2 in a sample from a subject and a reference value representing a certain prediction, diagnosis and/or prognosis of a given disease or condition as taught herein, the absence of such deviation is indicative of or may be attributed to the conclusion that the prediction, diagnosis and/or prognosis of said disease or condition in said subject is substantially the same as that represented by the reference value.

The present invention further provides kits or devices for diagnosing, predicting, prognosticating and/or monitoring of any one disease or condition as taught herein comprising means for detecting the level of the LTBP2 marker in a sample of the patient.

In a more preferred embodiment, such a kit or kits of the invention can be used in clinical settings or at home. The kit according to the invention can be used for diagnosing said disease or condition, for monitoring the effectiveness of treatment of a subject suffering from said disease or condition with an agent, or for preventive screening of subjects for the occurrence of said disease or condition in said subject.

In a clinical setting, the kit or device can be in the form of a bed-side device or in an emergency team setting, e.g. as part of the equipment of an ambulance or other moving emergency vehicle or team equipment or as part of a first-aid kit. The diagnostic kit or device can assist a medical practitioner, a first aid helper, or nurse to decide whether the patient under observation is developing an acute heart failure, after which appropriate action or treatment can be performed.

A home-test kit gives the patient a readout which he can communicate to a medicinal practitioner, a first aid helper or to the emergency department of a hospital, after which appropriate action can be taken. Such a home-test device is of particular interest for people having either a history of, or are at risk of suffering from any one disease or condition as taught herein or have a history or are at risk of suffering from dyspnea. Such subjects with a high risk for a disease or condition as taught herein or having a history of dyspnea could certainly benefit from having a home test device or kit according to the invention at home, inter alia because they can then easily distinguish between a renal dysfunction event and another event causing the dyspnea, resulting in an easier way of determining the actions to be taken to resolve the problem.

In said kit of the invention, the means or device for measuring the amount of the LTBP2 marker in said sample (b) can be any means or device that can specifically detect the amount of the LTBP2 protein in the sample. Examples are systems comprising LTBP2 specific binding molecules attached to a solid phase, e.g. lateral flow strips or dipstick devices and the like well known in the art. One non-limiting example to perform a biochemical assay is to use a test-strip and labelled antibodies which combination does not require any washing of the membrane. The test strip is well known, for example, in the field of pregnancy testing kits where an anti-hCG antibody is present on the support, and is carried complexed with hCG by the flow of urine onto an immobilised second antibody that permits visualisation. Other non-limiting examples of such home test devices, systems or kits can be found for example in the following U.S. Pat. No. 6,107,045, U.S. Pat. Nos. 6,974,706, 5,108,889, 6,027,944, 6,482,156, 6,511,814, 5,824,268, 5,726,010, 6,001,658 or U.S. patent applications: 2008/0090305 or 2003/0109067.

In a preferred embodiment, the invention provides a lateral flow device or dipstick. Such dipstick comprises a test strip allowing migration of a sample by capillary flow from one end of the strip where the sample is applied to the other end of such strip where presence of an analyte in said sample is measured.

In another embodiment, the invention provides a device comprising a reagent strip. Such reagent strip comprises one or more test pads which when wetted with the sample, provide a color change in the presence of an analyte and/or indicate the concentration of the protein in said sample.

In order to obtain a semi-quantitative test strip in which only a signal is formed once the LTBP2 protein level in the sample is higher than a certain predetermined threshold level or value, the reaction zone (5) comprising the non-fixed conjugated LTBP2 binding molecules, could also comprise a predetermined amount of fixed LTBP2 capture antibodies. This enables to capture away a certain amount of LTBP2 protein present in the sample, corresponding to the threshold level or value as predetermined. The remaining amount of LTBP2 protein (if any) bound by the conjugated or labelled binding molecules can then be allowed to migrate to the detection zone (6). In this case, the reaction zone (6) will only receive labelled binding molecule-LTBP2 complexes and subsequently only produce a signal if the level of the LTBP2 protein in the sample is higher than the predetermined threshold level or value.

Another possibility to determine whether the amount of the LTBP2 protein in the sample is below or above a certain threshold level or value, is to use a primary capturing antibody capturing all LTBP2 protein present in the sample, in combination with a labeled secondary antibody, developing a certain signal or color when bound to the solid phase. The intensity of the color or signal can then either be compared to a reference color or signal chart indicating that when the intensity of the signal is above a certain threshold signal, the test is positive (i.e. renal dysfunction or kidney failure is imminent). Alternatively, the amount or intensity of the color or signal can be measured with an electronic device comprising e.g. a light absorbance sensor or light emission meter, resulting in a numerical value of signal intensity or color absorbance formed, which can then be displayed to the subject in the form of a negative result if said numerical value is below the threshold value or a positive result if said numerical value is above the threshold value. This embodiment is of particular relevance in monitoring the LTBP2 level in a patient over a period of time.

The reference value or range can e.g. be determined using the home device in a period wherein the subject is free of a given disease or condition, giving the patient an indication of his base-line LTBP2 level. Regularly using the home test device will thus enable the subject to notice a sudden change in LTBP2 levels as compared to the base-line level, which can enable him to contact a medical practitioner.

Alternatively, the reference value can be determined in the subject suffering from a given disease or condition as taught herein, which then indicates his personal LTBP2 “risk level”, i.e. the level of LTBP2 which indicates he is or will soon be exposed to said disease or condition. This risk level is interesting for monitoring the disease progression or for evaluating the effect of the treatment. Reduction of the LTBP2 level as compared to the risk level indicates that the condition of the patient is improving.

Furthermore, the reference value or level can be established through combined measurement results in subjects with highly similar disease states or phenotypes (e.g. all having no disease or condition as taught herein or having said disease or condition).

Non-limiting examples of such semi-quantitative tests known in the art, the principle of which could be used for the home test device according to the present invention are the HIV/AIDS test or Prostate Cancer tests sold by Sanitoets. The home prostate test is a rapid test intended as an initial semi-quantitative test to detect PSA blood levels higher than 4 ng/ml in whole blood. The typical home self-test kit comprises the following components: a test device to which the blood sample is to be administered and which results in a signal when the protein level is above a certain threshold level, an amount of diluent e.g. in dropper pipette to help the transfer of the analytes (i.e. the protein of interest) from the sample application zone to the signal detection zone, optionally an empty pipette for blood specimen collection, a finger pricking device, optionally a sterile swab to clean the area of pricking and instructions of use of the kit.

Similar tests are also known for e.g. breast cancer detection and CRP-protein level detection in view of cardiac risk home tests. The latter test encompasses the sending of the test result to a laboratory, where the result is interpreted by a technical or medical expert. Such telephone or internet based diagnosis of the patient's condition is of course possible and advisable with most of the kits, since interpretation of the test result is often more important than conducting the test. When using an electronic device as mentioned above which gives a numerical value of the level of protein present in the sample, this value can of course easily be communicated through telephone, mobile telephone, satellite phone, E-mail, internet or other communication means, warning a hospital, a medicinal practitioner or a first aid team that a person is, or may be at risk of, suffering from kidney failure. A non-limiting example of such a system is disclosed in U.S. Pat. No. 6,482,156.

The presence and/or concentration of LTBP2 in a sample can be measured by surface plasmon resonance (SPR) using a chip having LTBP2 binding molecule immobilized thereon, fluorescence resonance energy transfer (FRET), bioluminescence resonance energy transfer (BRET), fluorescence quenching, fluorescence polarization measurement or other means known in the art. Any of the binding assays described can be used to determine the presence and/or concentration of LTBP2 in a sample. To do so, LTBP2 binding molecule is reacted with a sample, and the concentration of LTBP2 is measured as appropriate for the binding assay being used. To validate and calibrate an assay, control reactions using different concentrations of standard LTBP2 and/or LTBP2 binding molecule can be performed. Where solid phase assays are employed, after incubation, a washing step is performed to remove unbound LTBP2. Bound, LTBP2 is measured as appropriate for the given label (e.g., scintillation counting, fluorescence, antibody-dye etc.). If a qualitative result is desired, controls and different concentrations may not be necessary. Of course, the roles of LTBP2 and LTBP2 binding molecule may be switched; the skilled person may adapt the method so LTBP2 binding molecule is applied to sample, at various concentrations of sample.

A LTBP2 binding molecule according to the invention is any substance that binds specifically to LTBP2. Examples of a LTBP2 binding molecule useful according to the present invention, includes, but is not limited to an antibody, a polypeptide, a peptide, a lipid, a carbohydrate, a nucleic acid, peptide-nucleic acid, small molecule, small organic molecule, or other drug candidate. A LTBP2 binding molecule can be natural or synthetic compound, including, for example, synthetic small molecule, compound contained in extracts of animal, plant, bacterial or fungal cells, as well as conditioned medium from such cells. Alternatively, LTBP2 binding molecule can be an engineered protein having binding sites for LTBP2. According to an aspect of the invention, a LTBP2 binding molecule binds specifically to LTBP2 with an affinity better than 10⁻⁶ M. A suitable LTBP2 binding molecule e can be determined from its binding with a standard sample of LTBP2. Methods for determining the binding between LTBP2 binding molecule and LTBP2 are known in the art. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein. Such antibody may be commercially available antibody against LTBP2, such as, for example, a mouse, rat, human or humanised monoclonal antibody.

In a preferred embodiment, the binding molecule or agent is capable of binding both the mature membrane- or cell-bound LTBP2 protein or fragment. In a more preferred embodiment, the binding agent or molecule is specifically binding or detecting the soluble form, preferably the plasma circulating form of LTBP2, as defined herein.

According to one aspect of the invention, the LTBP2 binding molecule is labelled with a tag that permits detection with another agent (e.g. with a probe binding partner). Such tags can be, for example, biotin, streptavidin, his-tag, myc tag, maltose, maltose binding protein or any other kind of tag known in the art that has a binding partner. Example of associations which can be utilised in the probe:binding partner arrangement may be any, and includes, for example biotin:streptavidin, his-tag:metal ion (e.g. Ni²⁺), maltose:maltose binding protein.

The specific-binding agents, peptides, polypeptides, proteins, biomarkers etc. in the present kits may be in various forms, e.g., lyophilised, free in solution or immobilised on a solid phase. They may be, e.g., provided in a multi-well plate or as an array or microarray, or they may be packaged separately and/or individually. The may be suitably labelled as taught herein. Said kits may be particularly suitable for performing the assay methods of the invention, such as, e.g., immunoassays, ELISA assays, mass spectrometry assays, and the like.

The term “modulate” generally denotes a qualitative or quantitative alteration, change or variation specifically encompassing both increase (e.g., activation) or decrease (e.g., inhibition), of that which is being modulated. The term encompasses any extent of such modulation.

For example, where modulation effects a determinable or measurable variable, then modulation may encompass an increase in the value of said variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of said variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation.

Preferably, modulation of the activity and/or level of intended target(s) (e.g., LTBP2 gene or protein) may be specific or selective, i.e., the activity and/or level of intended target(s) may be modulated without substantially altering the activity and/or level of random, unrelated (unintended, undesired) targets.

Reference to the “activity” of a target such as LTBP2 protein may generally encompass any one or more aspects of the biological activity of the target, such as without limitation any one or more aspects of its biochemical activity, enzymatic activity, signalling activity and/or structural activity, e.g., within a cell, tissue, organ or an organism.

In the context of therapeutic or prophylactic targeting of a target, the reference to the “level” of a target such LTBP2 gene or protein may preferably encompass the quantity and/or the availability (e.g., availability for performing its biological activity) of the target, e.g., within a cell, tissue, organ or an organism.

For example, the level of a target may be modulated by modulating the target's expression and/or modulating the expressed target. Modulation of the target's expression may be achieved or observed, e.g., at the level of heterogeneous nuclear RNA (hnRNA), precursor mRNA (pre-mRNA), mRNA or cDNA encoding the target. By means of example and not limitation, decreasing the expression of a target may be achieved by methods known in the art, such as, e.g., by transfecting (e.g., by electroporation, lipofection, etc.) or transducing (e.g., using a viral vector) a cell, tissue, organ or organism with an antisense agent, such as, e.g., antisense DNA or RNA oligonucleotide, a construct encoding the antisense agent, or an RNA interference agent, such as siRNA or shRNA, or a ribozyme or vectors encoding such, etc. By means of example and not limitation, increasing the expression of a target may be achieved by methods known in the art, such as, e.g., by transfecting (e.g., by electroporation, lipofection, etc.) or transducing (e.g., using a viral vector) a cell, tissue, organ or organism with a recombinant nucleic acid which encodes said target under the control of regulatory sequences effecting suitable expression level in said cell, tissue, organ or organism. By means of example and not limitation, the level of the target may be modulated via alteration of the formation of the target (such as, e.g., folding, or interactions leading to formation of a complex), and/or the stability (e.g., the propensity of complex constituents to associate to a complex or disassociate from a complex), degradation or cellular localisation, etc. of the target.

The term “antisense” generally refers to a molecule designed to interfere with gene expression and capable of specifically binding to an intended target nucleic acid sequence. Antisense agents typically encompass an oligonucleotide or oligonucleotide analogue capable of specifically hybridising to the target sequence, and may typically comprise, consist essentially of or consist of a nucleic acid sequence that is complementary or substantially complementary to a sequence within genomic DNA, hnRNA, mRNA or cDNA, preferably mRNA or cDNA corresponding to the target nucleic acid. Antisense agents suitable herein may typically be capable of hybridising to their respective target at high stringency conditions, and may hybridise specifically to the target under physiological conditions.

The term “ribozyme” generally refers to a nucleic acid molecule, preferably an oligonucleotide or oligonucleotide analogue, capable of catalytically cleaving a polynucleotide. Preferably, a “ribozyme” may be capable of cleaving mRNA of a given target protein, thereby reducing translation thereof. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484).

“RNA interference” or “RNAi” technology is routine in the art, and suitable RNAi agents intended herein may include inter alia short interfering nucleic acids (siNA), short interfering RNA (sRNA), double-stranded RNA (dsRNA), micro-RNA (miRNA), and short hairpin RNA (shRNA) molecules as known in the art. For teaching on RNAi molecules and design thereof, see inter alia Elbashir et al. 2001 (Nature 411: 494-501), Reynolds et al. 2004 (Nat Biotechnol 22: 326-30), Wang & Mu 2004 (Bioinformatics 20: 1818-20), Yuan et al. 2004 (Nucleic Acids Res 32(Web Server issue): W130-4), by M Sohail 2004 (“Gene Silencing by RNA Interference: Technology and Application”, 1^{st} ed., CRC, ISBN 0849321417), U Schepers 2005 (“RNA Interference in Practice: Principles, Basics, and Methods for Gene Silencing in C. elegans, Drosophila, and Mammals”, 1^{st} ed., Wiley-VCH, ISBN 3527310207), and D R Engelke & J J Rossi 2005 (“Methods in Enzymology, Volume 392: RNA Interference”, 1^{st} ed., Academic Press, ISBN 0121827976).

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

The present active substances (agents) may be used alone or in combination with any therapies known in the art for the disease and conditions as taught herein (“combination therapy”). Combination therapies as contemplated herein may comprise the administration of at least one active substance of the present invention and at least one other pharmaceutically or biologically active ingredient. Said present active substance(s) and said pharmaceutically or biologically active ingredient(s) may be administered in either the same or different pharmaceutical formulation(s), simultaneously or sequentially in any order.

The dosage or amount of the present active substances (agents) used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every two or three weeks.

As used herein, a phrase such as “a subject in need of treatment” includes subjects that would benefit from treatment of a given disease or condition as taught herein. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, as well as prophylactic or preventative measures, wherein the aim is to prevent or lessen the chances of incidence of an undesired affliction, such as to prevent the chances of contraction and progression of a disease or condition as taught herein. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present compounds.

The above aspects and embodiments are further supported by the following non-limiting examples.

### EXAMPLES

### Example 1

### MASSterclass Targeted Protein Quantitation

### MASSTERCLASS Experimental Setup

MASSterclass assays use targeted tandem mass spectrometry with stable isotope dilution as an end-stage peptide quantitation system (also called Multiple Reaction Monitoring (MRM) and Single Reaction Monitoring (SRM). The targeted peptide is specific (i.e., proteotypic) for the specific protein of interest. i.e., the amount of peptide measured is directly related to the amount of protein in the original sample. To reach the specificity and sensitivity needed for biomarker quantitation in complex samples, peptide fractionation precedes the end-stage quantitation step.

A suitable MASSTERCLASS assay may include the following steps:
Plasma/serum sample
Depletion of human albumin and IgG (complexity reduction on protein level) using affinity capture with anti-albumin and anti-IgG antibodies using ProteoPrep spin columns (Sigma Aldrich)
Spiking of known amounts of isotopically labelled peptides. These peptides has the same amino acid sequence as the proteotypic peptides of interest, typically with one isotopically labelled amino acid built in to generate a mass difference. During the entire process, the labelled peptide has identical chemical and chromatographic behaviour as the endogenous peptide, except during the end-stage quantitation step which is based on molecular mass.
Tryptic digest. The proteins in the depleted serum/plasma sample are digested into peptides using trypsin. This enzyme cleaves proteins C-terminally from lysine and argninine, except when a proline is present C-terminally of the lysine or arginine. Before digestion, proteins are denatured by boiling, which renders the protein molecule more accessible for the trypsin activity during the 16 h incubation at 37° C.
Peptide-based fractionation: The charged peptide molecules are separated based on their specific isoelectric property. As there is no pl difference between the endogenous peptide and the isotopically labelled variant, they co-elute. Only those fractions containing the monitored peptides, or pools thereof, are selected and proceed to the next level of fractionation.
LC-MS/MS based quantitation, including further separation on reversed phase (C18) nanoLC (PepMap C18; Dionex) and MS/MS: tandem mass spectrometry using MRM (4000 QTRAP; ABI) or SRM (Vantage TSQ; Thermo Scientific) mode. The LC column is connected to an electrospray needle connected to the source head of the mass spectrometer. As material elutes from the column, molecules are ionized and enter the mass spectrometer in the gas phase. The peptide that is monitored is specifically selected to pass the first quadrupole (Q1), based on its mass to charge ratio (m/z). The selected peptide is then fragmented in a second quadrupole (Q2) which is used as a collision cell. The resulting fragments then enter the third quadrupole (Q3). Depending on the instrument settings (determined during the assay development phase) only a specific peptide fragment or specific peptide fragments (or so called transitions) are selected for detection.
The combination of the m/z of the monitored peptide and the m/z of the monitored fragment of this peptide is called a transition. This process can be performed for multiple transitions during one experiment. Both the endogenous peptide (analyte) and its corresponding isotopically labelled synthetic peptide (internal standard) elute at the same retention time, and are measured in the same LC-MS/MS experiment.
The MASSterclass readout is defined by the ratio between the area under the peak specific for the analyte and the area under the peak specific for the synthetic isotopically labelled analogue (internal standard). MASSterclass readouts are directly related to the original concentration of the protein in the sample. MASSterclass readouts can therefore be compared between different samples and groups of samples.

A typical MASSTERCLASS protocol followed in the present study:
25 μL of plasma is subjected to a depletion of human albumin and IgG (ProteoPrep spin columns; Sigma Aldrich) according to the manufacturer's protocol, except that 20 mM NH₄HCO₃ was used as the binding/equilibration buffer.
The depleted sample (225 μL) is denatured for 15 min at 95° C. and immediately cooled on ice
2 pmol of each isotopically labeled peptide (custom made ‘Heavy AQUA’ peptide; Thermo Scientific) is spiked in the sample
20 μg trypsin is added to the sample and digestion is allowed for 16 h at 37° C.
Half of the resulting sample is applied to pl-based separation. Fractions containing the peptides of interest are pooled together, dried and resuspended in 0.1% formic acid.
20 μL of the final solution is separated using reverse-phase NanoLC with on-line MS/MS in SRM mode:
Column: PepMap C18, 75 μm I.D.×25 cm L, 100 Å pore diameter, 5 μm particle size
Solvent A: 0.1% formic acid
Solvent B: 80% acetonitrile, 0.1% formic acid
Gradient: 30 min; 2%-55% Solvent B
MS/MS in SRM mode: method contains the transitions for the analyte as well as for the synthetic, labeled peptide.
The used transitions were experimentally determined and selected during protein assay development

The unique peptide used for LTBP2 quantification: EQDAPVAGLQPVER (SEQ ID NO: 2)

The unique peptide used for Cystatin C quantification: ALDFAVGEYNK (SEQ ID NO: 3)

### Example 2

### Screening of Acute Dyspnea Samples for LTBP2

In this example the clinical utility of LTBP2 measurement for the evaluation of dyspneic patients was assessed.

The 299 clinical samples used in this study are part of the BASEL V cohort, a prospective study on consecutive patients presenting themselves to the ED of the university Hospital of BASEL with dyspnea as the most prominent symptom (part of this cohort is described in Potocki et al., Journal of Internal Medicine 2010 January; 267(1):119-29). The gold standard for the diagnosis of acute heart failure was based interpretation of two independent cardiologists of all medical records pertaining to the patient including 90 day follow up data and BNP levels. Based on this, 56% (n=168) of patients were adjudicated to have an acute heart failure event, others were classified as dyspnea non-heart failure. A wide range of clinical and marker variables was collected (for summary see Table 1) including patient demographics, medical history, chronic medication, renal function parameters, echo parameters, established cardiac and inflammatory marker levels. Glomerular filtration rate was calculated using the Modification of Diet in Renal Disease (MDRD) formula (Stevens et al., New England Journal of Medicine 2006; 354:2473-83). Patients were followed up for at least 1 year post admission to the hospital and all-cause-mortality was recorded.

LTBP2 and Cystatin C levels were measured using MASSterclass™ assays as described in Example 1. BNP, NT-proBNP and CRP levels were measured using commercially available immunoassays as described in Potocki et al (2010).

The diagnostic accuracy of a specific protein was determined by measuring the area under the Receiver-Operating-Characteristics (ROC) curves (AUC) as in Sullivan Pepe M (The statistical evaluation of medical tests for classification and prediction. 1993 Oxford University Press New York). The estimated and confidence intervals for AUCs were also computed using a non-parametric approach, namely bootstrapping (Efron B, Tibshirani R J. Nonparametric confidence intervals. An introduction to the bootstrap. Monographs on statistics and applied probability. 1993; 57:75-90 Chapman & Hall New York).

Associations of LTBP2, Cystatin C, BNP, NT-proBNP and CRP levels with all available clinical parameters were computed using univariate statistical tests. Spearman's ranked test was used to compute correlation coefficients and Wilcoxon rank sum test for assessing whether two independent samples of observation originate from the same population.

TABLE 1
Summary of patient characteristics included in the study

| | all patients |
|---|---|
| Characteristic | (n = 299) |
| age (yr) | 77 |
| gender (% male) | 52 |
| BMI | 26 |
| History (%) | |
| hypertension | 68 |
| heart failure | 24 |
| CAD | 28 |
| diabetes | 18 |
| COPD | 34 |
| chronic kidney | 28 |
| disease | |
| physical/ECG | |
| heart rate | 93 ± 23 |
| systolic bp | 138 ± 26 |
| diastolic bp | 83 ± 16 |
| LVEF | 24 (20-28) |
| lab s | |
| creatinin (umol/L) | 85 (66-120) |
| eGFR | 67 (44-89) |
| (mL/min/1.73 m2) | |
| BNP (pg/mL) | 350 (90-1120) |
| Nt-proBNP | 1656 (314-6105) |
| (pg/mL) | |
| diagnosis (%) | |
| ADHF | 56% |
| Pneumonia | 10% |
| Pulmonary | 3% |
| embolism | |
| COPD/Asthma | 16% |
| hyperventilation | 3% |
| other | 12% |
| outcome | |
| survival at 1 yr | 73% |

### Example 3

### LTBP2 as Predictive Marker for Mortality

In the cohort of acute dyspnea patients under study as described in Example 2, patients were followed up for at least one year post admission. At 1 year post admission, 82/299 subjects (27%) had died (all-cause mortality). The relation of LTBP2 and other clinical and marker variables to mortality was studied using different methods. Receiver-operator characteristic analysis with death at 1 year as the reference standard were performed and median area under the curve was calculated. Distributions of marker levels in “alive” and “death” patients were compared using the Wilcoxon rank-sum test. Kaplan Meier curves compared mortality rates across the follow-up period after presentation in groups divided as a function of LTBP2 levels.

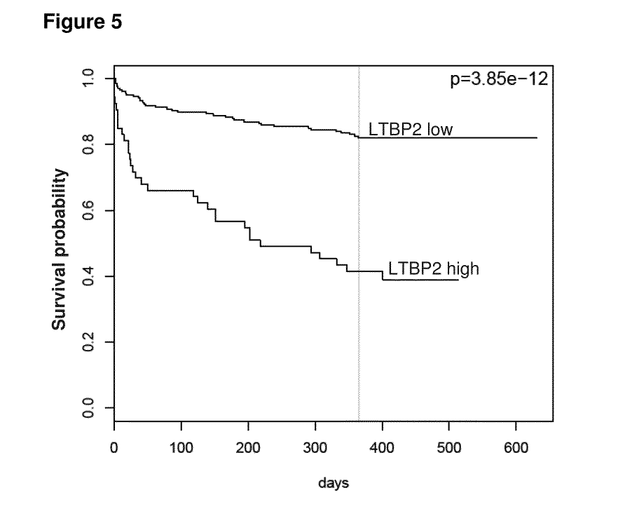

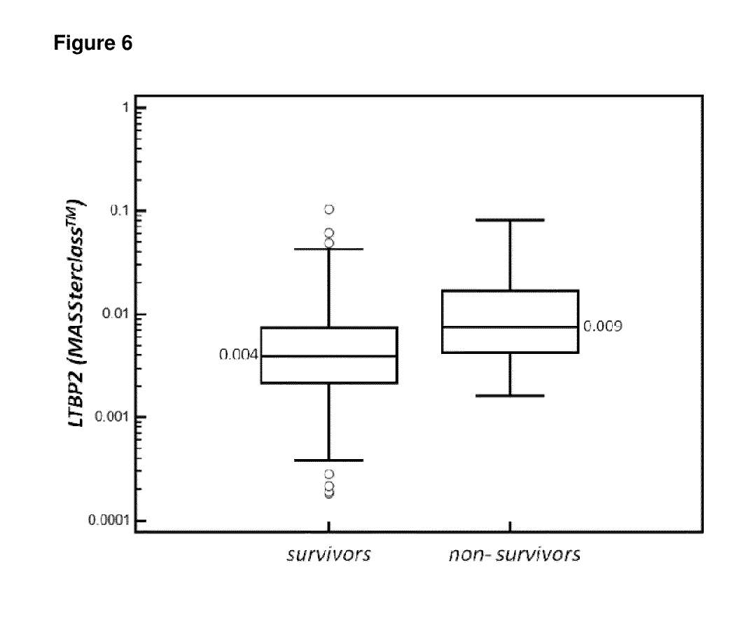

Concentrations of LTBP2 at presentation in patients with acute dyspnea were significantly higher among patients who died by 1 year (n=82; 27%) compared with patients who were alive (p=2e⁻¹¹) (FIG. 5A). This pattern of higher LTBP2 concentrations in decedents remained when subjects were considered as a function of the presence (p=3.5e⁻⁰⁸) or absence of acute heart failure (p=0.01) (FIG. 6A) and when the population was divided based on renal function (eGfr<60; p=8.8e-05 vs eGfr>60; p=0.0003) (FIG. 6B). This illustrates LTBP2 has the potential to predict bad outcome in a general dyspneic population as well as in an acute heart failure population and a chronic kidney disease population.

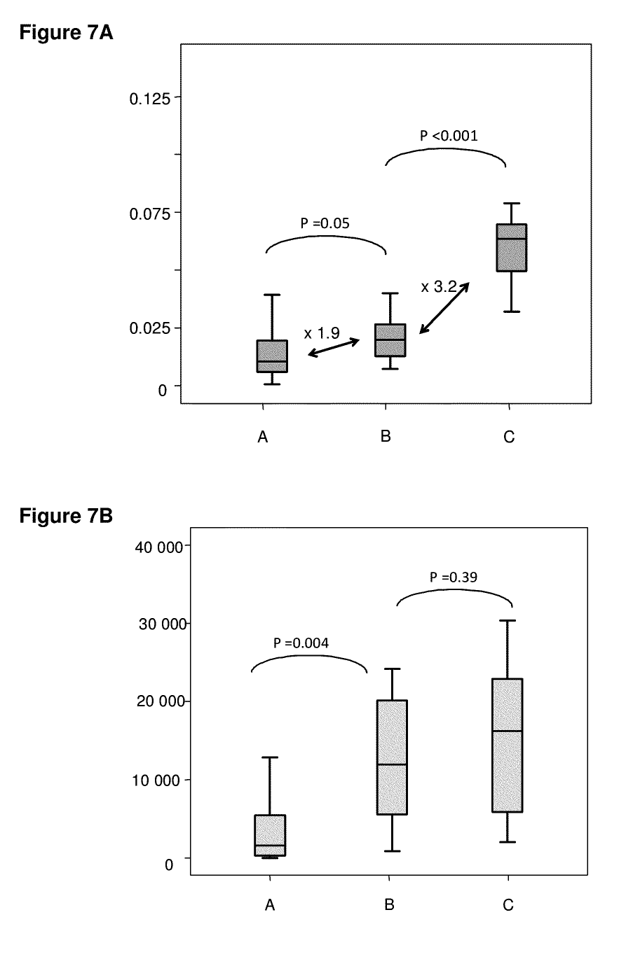

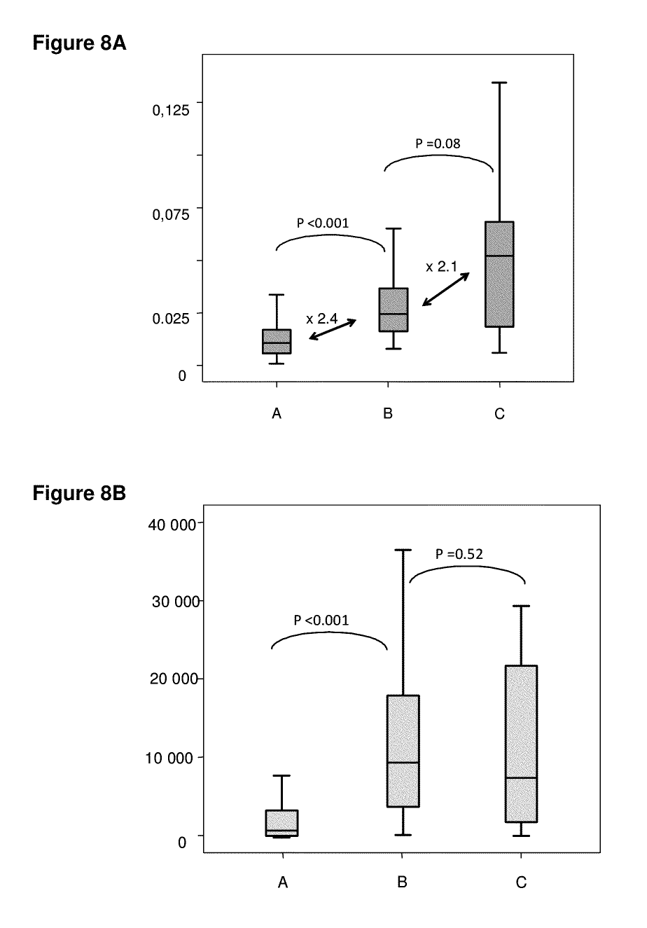

In addition decile analysis of LTBP2 concentrations examined as a function of mortality rates at 1 year revealed that there was a graded increase in mortality with rising concentrations of the marker (FIG. 5B). ROC analysis performed for predicting death at 1 year in all acute dyspnea patients demonstrated an AUC of 0.77 for LTBP2 (95% CI: 0.7-0.84), similar to NT-proBNP (AUC=0.77, but higher than BNP, Cystatin C and CRP protein markers (FIG. 7). Kaplan Meier analysis shows rates of death rise rapidly from admission up to 1 year for those patients with LTBP2 above the cut-off point at maximum accuracy (FIG. 8).

Multivariable Cox proportional hazard analysis using forward stepping were performed to identify the independent predictors of death at 1 year for this patient cohort. Variables were retained if their univariable p-value was <0.05 and entered into a multivariable model; hazard ratios (HR) were generated and only those variables with significant p values were retained in the final multivariable model. In this multivariate analysis LTBP2 levels above the cut off for maximum accuracy is a strong independent predictor of death at 1 yr in all dyspneic patients (HR=3.76; p<0.0001). Table 3 summarizes the selected univariable and multivariable predictors of 1 year mortality. Of note the final selected multivariable model contains LTBP2 combined with measures for renal function (eGfr and urea), bmi and potassium indicating LTBP2 can show complementarity over this variables for predicting survival.

TABLE 3
Selected univariable and multivariable predictors of 1-year mortality in dyspneic
patients (HR = hazard ratio; CI = confidence interval)

| | Univariable | | | Multivariable | | |
|---|---|---|---|---|---|---|
| Variable | HR | 95% CI | p-value | HR | 95% CI | p-value |
| age (yr) | 2.49 | 1.73-3.58 | <0.0001 | | | |
| admission weight | 0.73 | 0.55-0.96 | 0.0281 | | | |
| weight at discharge | 0.52 | 0.35-0.79 | 0.00169 | | | |
| admission bmi | 0.66 | 0.49-0.887 | 0.00569 | 0.55 | 0.4-0.768 | 0.000388 |
| admission systolic | 0.60 | 0.44-0.83 | 0.00167 | | | |
| blood pressure | | | | | | |
| admission diastolic | 0.72 | 0.54-0.98 | 0.0336 | | | |
| blood pressure | | | | | | |
| admission oxygen | 0.87 | 0.75-1.01 | 0.0629 | | | |
| saturation | | | | | | |
| admission oxygen | 1.51 | 1.31-1.75 | <0.0001 | | | |
| therapy | | | | | | |
| admission | 1.43 | 1.16-1.76 | 0.000832 | | | |
| respiratory rate | | | | | | |
| myoglobin ( | 1.83 | 1.44-2.32 | <0.0001 | | | |
| Potassium (mmol/L) | 1.19 | 1.12-1.26 | <0.0001 | 1.11 | 1.03-1.18 | 0.00374 |
| eGfr | 0.57 | 0.46-0.71 | <0.0001 | 1.72 | 1.02-2.9 | 0.0404 |
| (mL/min/1.73 m2) | | | | | | |
| Blood urea nitrogen | 2.51 | 1.91-3.29 | <0.0001 | 2.13 | 1.19-3.84 | 0.0112 |
| (mmol/L) | | | | | | |
| uric acid | 1.87 | 1.36-2.57 | 0.000121 | | | |
| albumin | 0.60 | 0.49-0.73 | <0.0001 | | | |
| hemoglobin (g/L) | 0.71 | 0.59-0.84 | <0.0001 | | | |
| hematocrit | 0.66 | 0.53-0.84 | 0.000487 | | | |
| LVEF (%) | 0.64 | 0.42-0.99 | 0.0467 | | | |
| Troponin T (ug/L) | 1.81 | 1.48-2.21 | <0.0001 | | | |
| Cystatin C | 2.20 | 1.65-2.92 | <0.0001 | | | |
| (MASSterclass | | | | | | |
| ratio) | | | | | | |
| LTBP2 | 3.46 | 2.47-4.85 | <0.0001 | 3.76 | 2.13-6.64 | <0.0001 |
| (MASSterclass | | | | | | |
| ratio) | | | | | | |
| BNP (pg/mL | 2.97 | 2.03-4.34 | <0.0001 | | | |
| NTproBNP (pg/mL) | 4.20 | 2.78-6.35 | <0.0001 | | | |
| CRP (mg/L) | 1.64 | 1.18-2.26 | 0.00279 | | | |

### Example 4

### Description of Patient Cohort of Systemic Inflamed Patients

Between 2004 and 2005, all patients with signs of systemic inflammatory response syndrome (SIRS) and suspicion of sepsis within the Utrecht Medical Center (Prof Verhoef, Utrecht, the Netherlands) were included in this study. A sample was taken for blood culture and at the same time a blood sample was collected for future biochemical analysis. In total over 1000 patients were included coming from different hospital departments. Final adjudicated diagnosis and classification as either SIRS or sepsis was done by three independent physicians based on all available clinical data (patient charts, culture of micro-organisms, biochemical markers, treatment and response to treatment, outcome). If left uncertain, the patient was called “possible sepsis”. SIRS, sepsis and severe sepsis definitions used were as set out in the sepsis guidelines (Levy et al., 2003), Center for Disease Control (CDC) criteria or as defined herein. Sepsis was defined as proven infection based on cultures (blood or other) or based on clinical presentation of the patient. Severe sepsis was defined as sepsis plus organ dysfunction. For each sepsis patient, the focus of primary infection was recorded and these were sub-grouped in respiratory tract, urogenital tract, gastro-intestinal tract or other. If other cultures than blood cultures were taken, this was recorded as well as the isolated micro-organism from the cultures. If antibiotics therapy was given, this was recorded, as well as whether the therapy turned out to be appropriate. For each patient the overall Sequential Organ Failure Assessment (SOFA) score was calculated based on the separate scores for respiratory, cardiovascular, hepatic, coagulation, renal and neurological systems. Finally the outcome (survivor versus non-survivor) at 28 days post day of blood sampling was recorded.

A subset of this database was used in this analysis. The set was sub-selected for community acquired infections (blood culture within 48 hrs of hospital admission) and patients with septic shock or under immune suppression and with uncertain final diagnosis were excluded. Table 4 summarizes the most important patient characteristics.

TABLE 4
Summary of patient characteristics

| | Survivor | Non-survivor |
|---|---|---|
| | (n = 311) | (n = 21) |
| Age | 55 | 67 |
| Gender (% male) | 51% | 48% |
| SOFA score | 1 (0-11) | 4 (0-8) |
| % sepsis | 73% | 60% |
| White blood cell count (×10⁹ cells/L) | 13.5 (0.2-45.9) | 13.9 (5.2-31.9) |
| CRP (ug/mL) | 95 (3-414) | 89 (7-422) |
| PCT (ug/mL) | 0.39 (0.02-223) | 0.61 (0.05-60.95) |

LTBP2 was measured using MASSterclass™ assays as described in Example 1. PCT and CRP were measured using commercially available immunoassays.

The diagnostic accuracy of a specific protein was determined by measuring the area under the Receiver-Operating-Characteristics (ROC) curves (AUC). (Sullivan Pepe M, The statistical evaluation of medical tests for classification and prediction, 1993, Oxford University Press New York). The estimated and confidence intervals for AUCs were also computed using a non-parametric approach, namely bootstrapping (Efron B, Tibshirani R J., Nonparametric confidence intervals. An introduction to the bootstrap. Monographs on statistics and applied probability, 1993; 57:75-90, Chapman & Hall New York).

### Example 5

### LTBP2 as a Marker for Prediction of Mortality in Patients Presenting with Signs of an Inflammatory Condition

In this cohort of patients with suspected sepsis, mortality at 28 days following blood culture was recorded. LTBP2 was examined for its performance to predict mortality in this patient set. Receiver operator characteristic (ROC) analysis showed LTBP2 has prognostic performance, better than other prognostic variables such as Procalcitonin, C-reactive protein (CRP), interleukine-6 (IL-6) and age (see Table 5). Box and whisker plots further illustrate LTBP2 levels are significantly elevated in patients which will die within 28 days compared to survivors (FIG. 15).

TABLE 5
AUC values of LTBP2 and other prognostic variables
such as Procalcitonin (CPT), C-reactive protein (CRP),
interleukine-6 (IL-6) and age

| Variable | AUC (95CI) |
|---|---|
| LTBP2 | 0.70 (0.57-0.83) |
| Procalcitonin (CPT) | 0.63 (0.51-0.75) |
| CRP | 0.45 (0.32-0.58) |
| IL-6 | 0.51 (0.40-0.61) |
| age | 0.66 (0.60-0.71) |

### Example 6

### LTBP2 as a Biomarker for Pulmonary Death in Patients with Acute Dyspnea

### Study Population

The study population consisted of unselected patients presenting to the emergency department of the University Hospital of Basel, Switzerland, with a chief complaint of acute dyspnea. From April 2006 to March 2007, 292 patients (out of 327 patients screened) were prospectively enrolled. Exclusion criteria were age younger than 18 years, an obvious traumatic cause of dyspnea and patients on haemodialysis. The study was carried out according to the principles of the Declaration of Helsinki and approved by the local ethics committee. Written informed consent was obtained from all participating patients.

### Clinical Evaluation and Follow-Up

Patients underwent an initial clinical assessment including clinical history, physical examination, electrocardiogram, pulse oximetry, blood tests including BNP, and chest X-ray. Echocardiography, pulmonary function tests and other diagnostic tests like CT-angiography were performed according to the treating physician. CT-angiography was the imaging modality of choice in patients with suspected pulmonary embolism. To assess the dyspnea severity we used the NYHA (New York Heart Association) functional classification with NYHA II as “dyspnea while walking up a slight incline”, III as “dyspnea while walking on level ground” and IV as “dyspnea at rest”.

Two independent internists blinded to LTBP2 reviewed all medical records including BNP levels and independently classified the patient's primary diagnosis into seven categories: acute heart failure (AHF), acute exacerbation of chronic obstructive pulmonary disease, pneumonia, acute complications of malignancy, acute pulmonary embolism, hyperventilation, and others. The two internists also independently adjudicated the cause of death. In the event of diagnostic disagreement among the internist reviewers, they were asked to meet to come to a common conclusion. In the event that they were unable to come to a common conclusion, a third-party internist adjudicator was asked to review the data and determine which diagnosis and cause of death was the most accurate.

The endpoint of the present study was 30-day cause specific mortality. 30-day all-cause mortality, one-year cause specific mortality and one-year all cause mortality were assessed as secondary endpoints. Cardiac death was defined as death due to coronary artery disease, heart failure or arrhythmias. Pulmonary death was defined as death due to acute exacerbations of chronic obstructive pulmonary disease, pneumonia and asthma. Each patient was contacted for follow-up, via telephone, by a single trained researcher after 365 days. In case the patient could not be reached referring physicians and relatives were contacted or the administrative databases of respective hometowns were reviewed to assess the survival status. Of note, one patient was lost to follow-up, so mortality analyses were performed in 291 patients.

### Laboratory Measurements

Blood samples for determination of LTBP2, BNP and NT-proBNP were collected at presentation into tubes containing potassium EDTA. After centrifugation, samples were frozen at −80° C. until assayed in a blinded fashion in a single batch. NT-proBNP levels were determined in a blinded fashion by a quantitative electrochemiluminescence immunoassay with CVs claimed by the manufacturer were 1.8% to 2.7% and 2.35% to 3.2% for within-run and total imprecision, respectively (Elecsys proBNP, Roche Diagnostics AG, Zug, Switzerland) and BNP was measured by a microparticle enzyme immunoassay at the hospital laboratory with a CVs claimed by the manufacturer of 4.3% to 6.3% and 6.5% to 9.4% for within-run and total imprecision, respectively. (AxSym, Abbott Laboratories, Abbott Park/IL, USA).

### Statistical Analysis

Continuous variables are presented as mean±SD or median (with interquartile range), and categorical variables as numbers and percentages. Univariate data on demographic and clinical features were compared by Mann-Whitney U test or Fisher's exact test as appropriate. Correlations among continuous variables were assessed by the Spearman rank-correlation coefficient. Receiver operating characteristic (ROC) curves were utilized to evaluate the accuracy of LTBP2, NT-proBNP and BNP to predict death. Areas under the curve (AUCs) were calculated for all markers. AUCs were compared according to the method by Hanley and McNeil. Cox regression analysis was assessed by univariate and multivariate analysis to identify independent predictors of outcome. Multivariable analysis, included all significant candidate variables (p<0.05) established in univariate analysis. The Kaplan-Meier cumulative survival curves were compared by the log-rank test. Glomerular filtration rate was calculated using the abbreviated Modification of Diet in Renal Disease (MDRD) formula. Data were statistically analysed with SPSS 15.0 software (SPSS Inc, Chicago, Ill., USA) and the MedCalc 9.3.9.0 package (MedCalc Software, Mariakerke, Belgium). All probabilities were two tailed and p<0.05 was regarded as significant.

### Patient Characteristics

The baseline characteristics of the 292 patients presenting with acute dyspnea are described in Table 1. Overall, mean age was 74±12 years (median 77 years, interquartile range (IQR) 68-83 years), 52% were men and 80% were in NYHA functional class III and IV. The primary diagnosis was AHF in 158 (54%) patients, acute exacerbation of chronic obstructive pulmonary disease in 57 (20%) patients, pneumonia in 32 (11%) patients, acute pulmonary embolism in 8 (3%) patients, acute complications of malignancy in 7 (2%) patients, hyperventilation in 5 (2%) patients, and other causes such as interstitial lung disease, asthma, or bronchitis in 24 (8%) patients.

TABLE 1
Baseline characteristics divided in patients with and without acute heart failure (AHF)

| Characteristic | Total (n = 292) | | AHF (n = 158) | | No AHF (n = 134) | | P-value |
|---|---|---|---|---|---|---|---|
| Age (years)^{a} | 74 ± 12 | | 78 ± 9 | | 68 ± 13 | | <0.0001 |
| Male sex (% of patients) | 52 | | 51 | | 53 | | 0.906 |
| BMI (kg/m²)^{a} | 26.1 ± 6.2 | | 26.6 ± 5.9 | | 25.5 ± 6.5 | | 0.124 |
| Medical conditions (% of | | | | | | | |
| patients) | | | | | | | |
| Heart failure | 24 | | 40 | | 7 | | <0.0001 |
| Coronary artery disease | 28 | | 38 | | 16 | | <0.0001 |
| Chronic obstructive | 34 | | 27 | | 42 | | 0.006 |
| pulmonary disease | | | | | | | |
| Diabetes | 18 | | 24 | | 11 | | 0.005 |
| Hypertension | 68 | | 78 | | 56 | | <0.0001 |
| Hyperlipidemia | 29 | | 33 | | 25 | | 0.165 |
| Chronic kidney disease | 28 | | 44 | | 11 | | <0.0001 |
| Initial clinical findings | | | | | | | |
| Heart rate (bpm)^{a} | 93 ± 23 | | 93 ± 25 | | 92 ± 19 | | 0.495 |
| Systolic pressure (mmHg)^{a} | 138 ± 26 | | 135 ± 27 | | 140 ± 25 | | 0.098 |
| NYHA functional class (% | | | | | | | |
| of patients) | | | | | | | |
| II | 20 | | 10 | | 32 | | <10.0001 |
| III | 40 | | 45 | | 35 | | 0.109 |
| IV | 40 | | 45 | | 33 | | 0.034 |
| Edema | 42 | | 57 | | 26 | | <0.0001 |
| Rales | 54 | | 64 | | 43 | | <0.0001 |
| Medication at admission | | | | | | | |
| Beta-blockers | 39 | | 57 | | 17 | | <0.0001 |
| ACE-Inhibitors/AT-receptor- | 49 | | 62 | | 34 | | <0.0001 |
| blockers | | | | | | | |
| Diuretics | 52 | | 64 | | 39 | | <0.0001 |
| Laboratory findings | | | | | | | |
| eGFR - ml/min/1.73 m2^{b} | 67 | [44-89] | 54 | [36-73] | 80 | [63-112] | <0.0001 |
| BNP (pmol/l)^{b} | 349 | [89-1121] | 976 | [467-1925] | 81 | [39-181] | <0.0001 |
| NT-proBNP (pmol/l)^{b} | 1656 | [314-6105] | 5757 | [1924-13243] | 300 | [76-974] | <0.0001 |

^{a}mean ± SD,
^{b}median (IQR = interquartile range), BMI = Body mass index; eGRF = estimated glomerular filtration rate; NYHA = New York Heart Association; BNP = B-type natriuretic peptide; NT-proBNP = N-teriminal pro-B-type natriuretic peptide

LTBP2 concentrations at presentation in patients with dyspnea were strongly correlated to markers of kidney dysfunction (creatinine: r=0.71, p<0.001; cystatin C: r=0.83, p<0.001), BNP (r=0.52, p<0.001) and NT-proBNP (r=0.66, p<0.001). Weaker albeit significant correlations existed with NYHA functional classes (r=0.18, p=0.003) and markers of infection (neutrophile count: r=0.23, p<0.001; C-reactive protein: r=0.13, p=0.04). These correlations were independent of the primary cause of dyspnea and persisted in AHF and non-AHF patients.

### LTBP2 Levels and Prognostic Value of LTBP2 on Short-Term Outcome

At 30 days, 29 patients (10%) had died. Non-survivors had significantly higher LTBP2 levels than survivors in the overall population (p<0.001), the AHF subgroup (p<0.001) and patients with dyspnea of pulmonary origin (p=0.011) (FIG. 1A). As further shown in FIG. 1A, LTBP2 levels were especially elevated in patients dying of pulmonary causes (Survivors: 0.011 normalized level [0.006-0.021] vs. Cardiac death: 0.021 normalized level [0.012-0.028] vs. Pulmonary death: 0.066 normalized level [0.043-0.078]). Contrastingly and as shown in FIG. 1B, natriuretic peptide levels did not differ significantly between patients dying of cardiac or pulmonary causes (NT-proBNP: 11941 pg/ml [3338-20973] vs. 16195 pg/ml [4897-25909]; p=0.39).

Receiver operating characteristic curve analyses were performed to assess the potential of LTBP2 levels to predict all-cause short term mortality. The areas under the curve (AUC) to predict all-cause mortality are for LTBP2 (0.79; 95% CI 70-87), NT-proBNP (0.75; 95% CI 0.65-0.84) and BNP (0.62; 95% CI 0.51-0.73). Cause specific mortality was looked at as well. Receiver operating characteristic curve (ROC) analyses demonstrated an AUC of 0.95 (95% CI 0.91-0.98) for LTBP2 to predict 30 day pulmonary mortality, which was significantly higher than the AUCs observed for NT-proBNP (0.84; 95% CI 0.75-0.94) and BNP (0.63; 95% CI 0.48-0.77) for 30 day pulmonary mortality (p=0.04 and <0.001, respectively).

### LTBP2 Levels and Prognostic Value of LTBP2 on One-Year Outcome

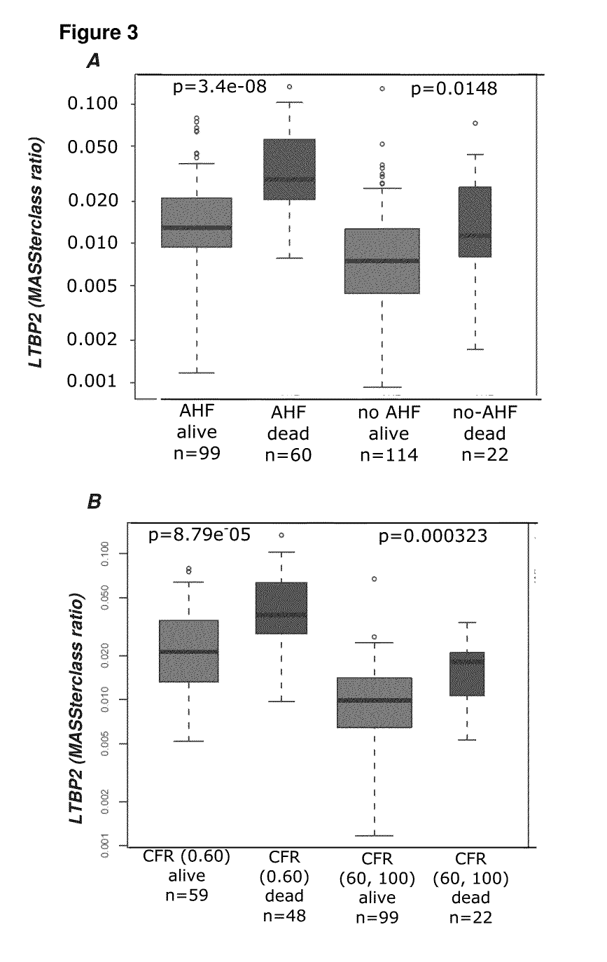

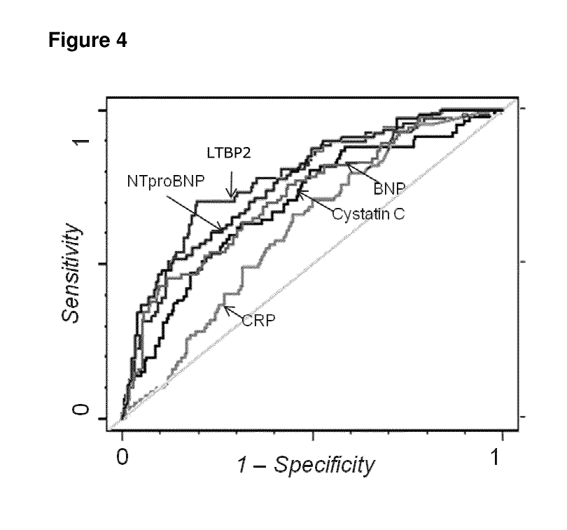

Overall 80 (27%) patients died during the first year of follow up; heart failure (n=28), myocardial infarction (n=14) and pulmonary death (n=14) were the most common causes of death. LTBP2 levels in non-survivors were significantly higher compared to survivors for the overall patient population (p<0.001), AHF patients (p<0.001) and non-AHF (p=0.021) patients. Again, there was a trend towards higher LTBP2 values in patients dying of pulmonary causes (Survivors: 0.01 normalized level [0.0056-0.016] vs. Cardiac death: 0.025 normalized level [0.016-0.037] vs. Pulmonary death: 0.052 normalized level [0.017-0.071]) (FIG. 3A). As shown in FIG. 3B, natriuretic peptide levels did not separate between causes of death (NT-proBNP 7785 pg/ml [1920-22584] vs. 9757 pg/ml [3772-18609]; p=0.52). Mortality according to LTBP2 level deciles is depicted in FIG. 4.

Receiver operating characteristic curve analyses were performed to assess the potential of LTBP2 levels to predict all-cause and cause specific one-year mortality. Importantly, the prognostic potential of LTBP2 (AUC 0.77; 95% CI 0.70-0.83) was comparable to NT-proBNP (AUC 0.77; 95% CI 0.71-0.84) and BNP (AUC 0.71; 95% CI 0.64-0.79) for the prediction of all-cause and cardiac mortality AUC 0.77, 0.79, 0.80, respectively) and tended to be superior for the prediction of pulmonary death AUC 0.80, 0.75, 0.59, respectively; p vs. NT-proBNP 0.59, p vs. BNP 0.04). Importantly, the predictive potential of LTBP2 was independent of kidney dysfunction and persisted in patients with preserved kidney function (AUC 0.77, 95% CI 0.70-0.83).

What is claimed is:

1. A method for treating a subject having increased risk of death within one year, said subject presenting with one or more signs of an inflammatory condition which is sepsis or SIRS, said method comprising the steps of:
(i) having a biological sample obtained from the subject;
(ii) having an assay conducted which comprises measuring the quantity of LTBP2 in said sample using an immunoassay, wherein the immunoassay employs an aptamer and/or antibody specifically binding to LTBP2, or using a binding agent capable of specifically binding to LTBP2 with an affinity better than 10⁻⁶ M;
(iii) comparing the quantity of LTBP2 measured in (ii) with a reference value of the quantity of LTBP2, said reference value representing a known risk of death within one year for a subject having the inflammatory condition;
(iv) predicting an increased risk of death in the subject if the quantity of LTBP2 measured in (ii) substantially corresponds to a reference value representing a subject having the inflammatory condition with increased risk of death within one year or if the quantity of LTBP2 measured in (ii) is elevated compared with the reference value
(v) treating the subject identified as having an increased risk of death in (iv) for the inflammatory condition.

2. The method according to claim 1, wherein the binding agent capable of specifically binding to LTBP2 is an aptamer or antibody specifically binding to LTBP2.

3. The method according to claim 1, wherein the subject presenting with one or more signs of an inflammatory condition has sepsis.

4. The method according to claim 1, wherein the subject presenting with one or more signs of an inflammatory condition has pulmonary inflammation.

5. The method according to claim 1, wherein the subject presenting with one or more signs of an inflammatory condition has acute dyspnea, acute heart failure, or renal dysfunction.

6. The method according to claim 1, for evaluating the risk of death within about 6 months, within about 5 months, within about 4 months, within about 3 months, within about 2 months, or within about one month, preferably within about one month.

7. The method according to claim 1, wherein the method further comprises measuring the presence or absence and/or quantity of one or more other biomarkers useful for evaluating the risk of death in the sample from the subject.

8. The method according to claim 7, wherein said other biomarker is selected from the group consisting of ST-2, galectin-3, midregional pro-adrenomedullin, creatinine, Cystatin C, neutrophil gelatinase-associated lipocalin (NGAL), beta-trace protein, kidney injury molecule 1 (KIM-1), interleukin-18 (IL-18), B-type natriuretic peptide (BNP), pro-B-type natriuretic peptide (proBNP), amino terminal pro-B-type natriuretic peptide (NTproBNP) and C-reactive peptide, and fragments or precursors of any one thereof.

9. The method according to claim 1, wherein said sample is blood, serum, plasma or urine.

10. The method according to claim 1, wherein the subject is a critically ill patient.

11. The method according to claim 4, for assessing the risk of dying within one month from a pulmonary cause or complication in the subject.

12. The method of claim 1, wherein the treatment comprises administration of an agent that modulates the level or activity of LTBP2.

13. The method of claim 1, wherein the treatment comprises administration of an antibiotic.

14. A method for evaluating the risk of death within a year for a subject presenting with one or more signs of an inflammatory condition, said method comprising the steps of:
(i) receiving data representative of values of the quantity of LTBP2 in a sample from the subject;
(ii) accessing a data repository on a computer, said data repository comprising a reference value of the quantity of LTBP2, said reference value representing a known risk of death, preferably a known risk of death within a year for a subject having an inflammatory condition; and
(iii) comparing the data as received in (i) with the reference value in the data repository on the computer, thereby making an evaluation of the risk of death within a year for the subject.


SEQUENCE LISTING (3 sequences)

SEQ ID NO: 1 - protein, 1821 aa, Homo sapiens
MRPRTKARSPGRALRNPWRGFLPLTLALFVGAGHAQRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGASWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGEAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 2 - protein, 14 aa, Homo sapiens
EQDAPVAGLQPVER

SEQ ID NO: 3 - protein, 11 aa, Homo sapiens
ALDFAVGEYNK